(19) United States
(12) Patent Application Publication
(10) Pub. No.: US 2023/0310186 A1
(43) Pub. Date: Oct. 5, 2023

(54) STENT AND STENT GRAFT

(71) Applicant: Japan Lifeline Co., Ltd., Tokyo (JP)

(72) Inventors: Junya NAGANO, Tokyo (JP); Haruki OKA, Tokyo (JP)

(21) Appl. No.: 18/160,111

(22) Filed: Jan. 26, 2023

(30) Foreign Application Priority Data
Mar. 30, 2022 (JP) 2022056339

(51) Int. Cl. A61F 2/90; A61F 2/07

(52) U.S. Cl. CPC A61F 2/90; A61F 2/07

(57) ABSTRACT

A stent formed by weaving a wire into a tubular shape includes intersecting portions at which the wire intersects and interlocking portions at which the wire interlocks. In the stent, at least two stretch assisting columns are continuously disposed in a circumferential direction of the stent, each of the at least two stretch assisting columns including a larger number of the intersecting portions than the interlocking portions in an axial direction of the stent, and at least one contraction assisting column including a larger number of the interlocking portions than the intersecting portions in the axial direction is disposed. When the stent is equally divided in the circumferential direction by a plane including an axis of the stent and a first equally-divided region includes the at least two stretch assisting columns continuously disposed, a second equally-divided region includes a majority of the interlocking portions.

### TECHNICAL FIELD

The present disclosure relates to a stent and the like.

### BACKGROUND ART

A stent is a medical device formed by weaving a metal wire or the like into a tubular shape. The stent is inserted into a tubular organ in the body such as a blood vessel, a trachea, a digestive tract, a common bile duct, and a pancreatic duct, or a junction (inlet and outlet) thereof, or a passage formed in the body for diagnosis or treatment purpose (for example, a puncture passage from a stomach or a duodenal bulb to a common bile duct), so as to dilate a target site.

### CITATION LIST

### Patent Literature

Patent Literature 1: JP 2021-142319 A

### SUMMARY OF INVENTION

### Technical Problem

A stent or a stent graft is inserted into various sites in a human body and, for example, may be inserted into a curved aorta for the treatment of aortic aneurysm or aortic dissection. A stent disclosed in Patent Literature 1 has a stretchability that varies along an axial direction so as to be prevented from falling off in the axial direction. However, this configuration is not necessarily suitable for a curved tubular organ or the like such as an aorta.

The present disclosure has been made in view of the circumstances described above, and an object thereof is to provide a stent and the like suitable for a curved site.

### Solution to Problem

In order to solve the above-described problem, a stent according to an aspect of the present invention is a stent formed by weaving a wire into a tubular shape, the stent including intersecting portions at which the wire intersects and interlocking portions at which the wire interlocks. In the stent, at least two stretch assisting columns are continuously disposed in a circumferential direction of the stent, each of the at least two stretch assisting columns including a larger number of the intersecting portions than the interlocking portions in an axial direction of the stent, and at least one contraction assisting column including a larger number of the interlocking portions than the intersecting portions in the axial direction is disposed.

According to this aspect, the at least two continuously-disposed stretch assisting columns each of which includes a larger number of the intersecting portions can stretch relatively easily, and the contraction assisting column including a larger number of the interlocking portions can contract relatively easily, and thus the stent can be easily curved.

Another aspect of the present invention is a stent graft. The stent graft includes a stent formed by weaving a wire into a tubular shape, and a graft covering the stent, the stent including intersecting portions at which the wire intersects, and interlocking portions at which the wire interlocks. In the stent, at least two stretch assisting columns are continuously disposed in a circumferential direction of the stent, each of the at least two stretch assisting columns including a larger number of the intersecting portions than the interlocking portions in an axial direction of the stent, and at least one contraction assisting column including a larger number of the interlocking portions than the intersecting portions in the axial direction is disposed.

### Advantageous Effects of Invention

According to the present disclosure, a stent and the like suitable for a curved site can be provided.

### BRIEF DESCRIPTION OF DRAWINGS

FIGS. 1A-1C schematically illustrate an outline of thoracic endovascular aortic repair (TEVAR).

FIGS. 2A and 2B schematically illustrate an intersecting portion and an interlocking portion formed when a stent having a tubular shape is woven with a wire.

FIGS. 3A and 3B illustrate a stent according to a first embodiment.

FIG. 4 illustrates the stent according to the first embodiment.

FIGS. 5A and 5B illustrate the stent according to the first embodiment.

FIG. 6 illustrates the stent according to the first embodiment.

FIG. 7 illustrates the stent according to the first embodiment.

FIGS. 8A and 8B illustrate a stent according to a second embodiment.

FIG. 9 illustrates the stent according to the second embodiment.

FIGS. 10A and 10B illustrate the stent according to the second embodiment.

FIG. 11 illustrates the stent according to the second embodiment.

FIG. 12 illustrates the stent according to the second embodiment.

FIGS. 13A and 13B illustrate a stent according to a third embodiment.

FIG. 14 illustrates the stent according to the third embodiment.

FIGS. 15A and 15B illustrate the stent according to the third embodiment.

FIG. 16 illustrates the stent according to the third embodiment.

FIG. 17 illustrates the stent according to the third embodiment.

FIGS. 18A and 18B illustrate a stent according to a fourth embodiment.

FIG. 19 illustrates the stent according to the fourth embodiment.

FIGS. 20A and 20B illustrate the stent according to the fourth embodiment.

FIG. 21 illustrates the stent according to the fourth embodiment.

FIG. 22 illustrates the stent according to the fourth embodiment.

FIGS. 23A and 23B illustrate a stent according to a fifth embodiment.

FIG. 24 illustrates the stent according to the fifth embodiment.

FIGS. 25A and 25B illustrate the stent according to the fifth embodiment.

FIG. 26 illustrates the stent according to the fifth embodiment.

FIG. 27 illustrates the stent according to the fifth embodiment.

FIGS. 28A and 28B illustrate a stent according to a sixth embodiment.

FIG. 29 illustrates the stent according to the sixth embodiment.

FIGS. 30A and 30B illustrate the stent according to the sixth embodiment.

FIG. 31 illustrates the stent according to the sixth embodiment.

FIG. 32 illustrates the stent according to the sixth embodiment.

FIGS. 33A and 33B illustrate a stent according to a seventh embodiment.

FIG. 34 illustrates the stent according to the seventh embodiment.

### DESCRIPTION OF EMBODIMENTS

Hereinafter, modes (hereinafter also referred to as “embodiments”) for carrying out the present invention will be described in detail with reference to the drawings. In the description and/or drawings, the same or equivalent constituent elements, members, and processing operations, and the like are denoted by the same reference numerals, and redundant descriptions are omitted. The scales and shapes of the illustrated parts are set for convenience to simplify the explanation and should not be construed in a limited manner unless otherwise specified. The embodiments are illustrative and do not limit the scope of the present invention in any way. Not all features or combinations of the features described in the embodiments are essential to the present invention.

While the present disclosure can be applied to a stent and/or a stent graft inserted into any site in a human body, a stent graft inserted into an aorta for the treatment of aortic aneurysm or aortic dissection will be described as an example in the present embodiment. Specifically, a stent graft used for stent graft insertion in the thoracic aorta called thoracic endovascular aortic repair (TEVAR) will be described.

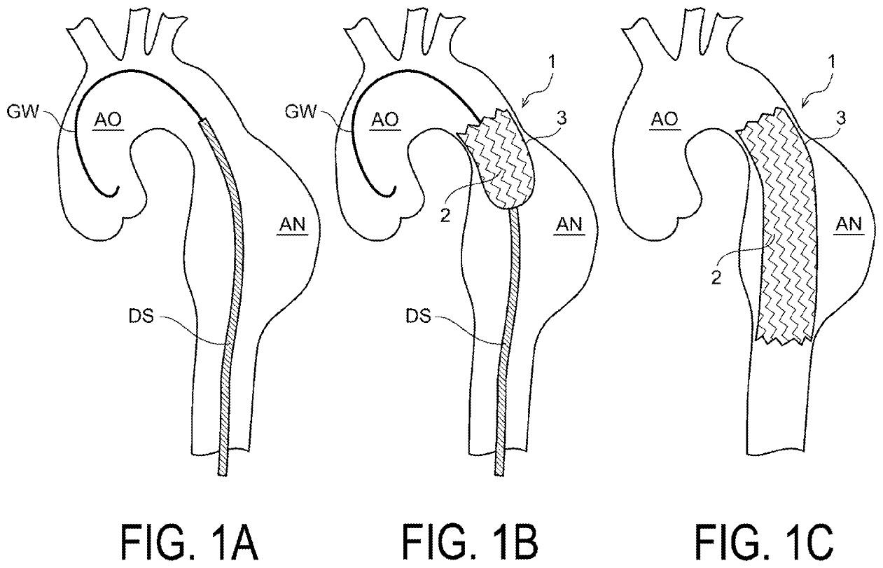

FIGS. 1A-1C schematically illustrate an outline of TEVAR. As illustrated in FIG. 1A, a delivery system DS is inserted into an aorta AO in which an aortic aneurysm AN is formed. Specifically, the distal end of the delivery system DS is inserted beyond the aortic aneurysm AN while being guided by a guide wire GW inserted in advance in the aorta AO. The delivery system DS includes an outer sheath configured to hold a stent graft 1 in a reduced-diameter state. That is, the stent graft 1 is housed in the outer sheath. In this state, only the outer sheath is pulled out while the stent graft 1 inside the outer sheath is left behind as illustrated in FIG. 1B. In the stent graft 1 after the removal of the outer sheath, a stent 2 formed of a wire of a metal material such as stainless steel or nickel-titanium alloy or a non-metal material expands together with a graft 3 which is made of a plastic material such as polyethylene terephthalate (PET) or polytetrafluoroethylene (PTFE) and covers the stent 2. As illustrated in FIG. 1C, the stent graft 1 placed in the aorta AO after the outer sheath is completely pulled out extends across the aortic aneurysm AN, and the graft 3 forms an artificial blood vessel directly connecting both sides of the aortic aneurysm AN. As a result, the blood inside the aorta AO flows through the graft 3 without flowing into the aortic aneurysm AN, and thus enlargement or rupture of the aortic aneurysm AN can be prevented.

As illustrated in FIGS. 1A-1C, the aorta AO is not perfectly straight, but is curved. The stent graft 1 according to the present embodiment to be described in detail below has a configuration suitable for a curved site such as the aorta AO. Specifically, a configuration is adopted in which a focus is placed on an aspect that the stent 2 can easily stretch at an intersecting portion at which the wire of the stent 2 intersects, and the stent 2 can easily contract at an interlocking portion at which the wire of the stent 2 interlocks. For example, a stretch assisting region including a larger number of the intersecting portions and a contraction assisting region including a larger number of the interlocking portions are separately disposed along the circumferential direction of the stent 2, and thus the stent graft 1 can be easily curved to fit a dilation target site having a curvature.

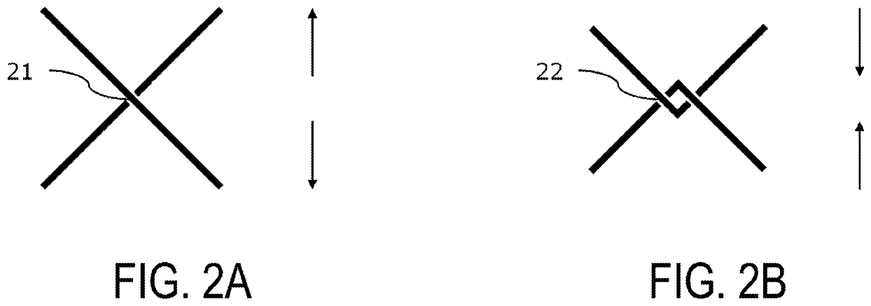

FIGS. 2A and 2B schematically illustrate an intersecting portion 21 and an interlocking portion 22 formed when the stent 2 having a tubular shape is woven with a wire. The wires forming the stent 2 having a tubular shape intersect at the intersecting portion 21 (hereinafter also referred to as a “crossing portion 21”) illustrated in FIG. 2A. Specifically, at the intersecting portion 21, two wires (or an identical wire folded back in the axial direction) pass each other with a shift in the radial direction of the stent 2 having a tubular shape (a direction perpendicular to the paper surface in FIGS. 2A and 2B). The wires forming the stent 2 having a tubular shape interlock at the interlocking portion 22 (hereinafter also referred to as a “hooking portion 22”) illustrated in FIG. 2B. Specifically, at the interlocking portion 22, two wires (or an identical wire folded back in the axial direction) are bent and hooked to each other, and thereby separation of the wires is restricted in the direction where the wires are hooked to each other along the axial direction of the stent 2 having a tubular shape (approaching of the wires is not restricted along the axial direction). Note that, in the following drawings, the interlocking portion 22 is simply illustrated as a rhomboid. The intersecting portion 21 is easy to stretch and hard to contract along the axial direction (the vertical direction in FIG. 2) of the stent 2 having a tubular shape. The interlocking portion 22 is easy to contract and hard to stretch along the axial direction of the stent 2 having a tubular shape. For this reason, when the stent graft 1 is placed at a dilation target site having a curvature, many hooking portions 22, which are easy to contract, are distributed on the inner side having a large curvature and many crossing portions 21, which are easy to stretch, are distributed on the outer side having a small curvature, so that the stent graft 1 can be easily curved.

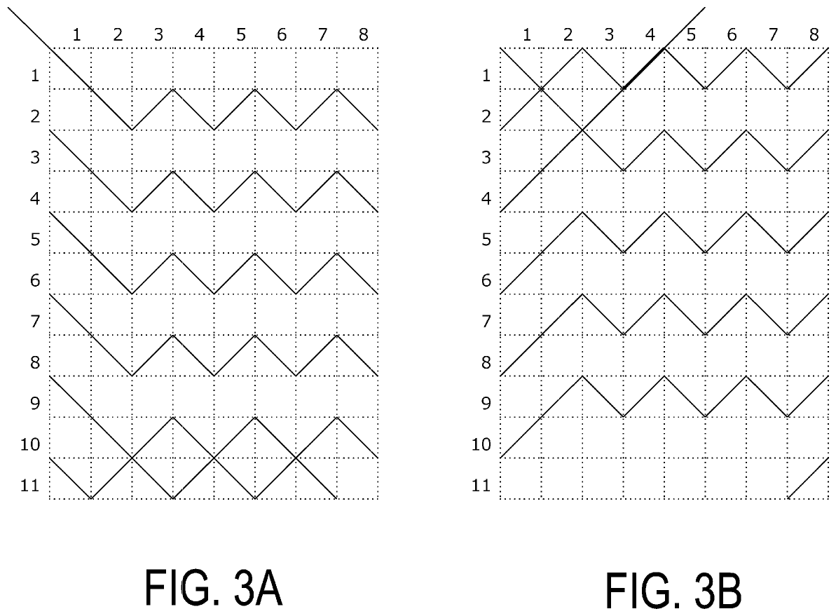

FIGS. 3A and 3B schematically illustrate a weaving method of a first wire forming the stent 2 according to the first embodiment. In this drawing and subsequent similar drawings, the horizontal direction represents the circumferential direction of the stent 2 having a tubular shape, and the vertical direction represents the axial direction of the stent 2 having a tubular shape. For convenience of illustration, the stent 2 is developed in the circumferential direction. That is, the left end and the right end of the stent 2 illustrated are continuous, forming a substantially circular circumference of the stent 2 having a tubular shape. The wire can form the intersecting portion 21 or the interlocking portion 22 at each grid point of an imaginary grid indicated by dotted lines. In this drawing and subsequent similar drawings, the position of each grid point in the circumferential direction (horizontal direction) is represented by an x coordinate, and the position of each grid point in the axial direction (vertical direction) is represented by a y coordinate. The total number of the grid points in the circumferential direction is X, and the total number of the grid points in the axial direction is Y. In the example of FIGS. 3A and 3B, X=8 and Y=11. For example, in FIG. 3A, the xy coordinates (x, y) of the grid point at the upper left corner (origin) are represented as (0, 0), the xy coordinates (x, y) of the grid point at the upper right corner are represented as (8, 0), the xy coordinates (x, y) of the grid point at the lower left corner are represented as (0, 11), and the xy coordinates (x, y) of the grid point at the lower right corner are represented as (8, 11). Note that in the actual stent 2, it is also assumed that the respective grid points are not necessarily aligned in the circumferential direction (x direction) and/or the axial direction (y direction). In such a case, the similarity to the present invention and/or the present embodiment is determined only after the respective grid points of the stent 2 are disposed in the grid pattern as illustrated in the drawing.

FIG. 3A illustrates a state in which the first wire forming the stent 2 is woven from a first end (the upper end) toward a second end (the lower end) in the axial direction. FIG. 3B illustrates a state in which, following FIG. 3A, the first wire is folded back at the second end (the lower end) and then woven from the second end (the lower end) toward the first end (the upper end) in the axial direction. That is, the first wire is woven back and forth in the axial direction, and FIG. 3A represents a forward path and FIG. 3B represents a backward path.

In FIG. 3A, the first wire brought in from a starting point (0, 0) goes to a rightmost grid point (8, 2) while bending at bending points (2, 2), (3, 1), (4, 2), (5, 1), (6, 2), and (7, 1) in sequence. As will be described later, the hooking portion 22 is formed at each bending point. Similarly thereafter, the first wire is woven from a leftmost grid point (0, 2), which is identical to the rightmost grid point (8, 2), toward a rightmost grid point (8, 4). The first wire is woven from a leftmost grid point (0, 4), which is identical to the rightmost grid point (8, 4), toward a rightmost grid point (8, 6). The first wire is woven from a leftmost grid point (0, 6), which is identical to the rightmost grid point (8, 6), toward a rightmost grid point (8, 8). The first wire is woven from a leftmost grid point (0, 8), which is identical to the rightmost grid point (8, 8), toward a rightmost grid point (8, 10).

Then, from a leftmost grid point (0, 10), which is identical to the rightmost grid point (8, 10), the first wire goes to a lowermost grid point or a folded-back point (7, 11) while bending at bending points (1, 11), (2, 10), (3, 11), (4, 10), (5, 11), and (6, 10) in sequence. Note that no hooking portions 22 are formed at bending points (1, 11), (3, 11), and (5, 11) and the folded-back point (7, 11) of the stent 2 located at the lower end.

In FIG. 3B, after being folded back at the folded-back point (7, 11), the first wire goes to the rightmost grid point (8, 10). Subsequently to the leftmost grid point (0, 10), which is identical to the rightmost grid point (8, 10), the first wire goes to the rightmost grid point (8, 8) while bending at bending points (2, 8), (3, 9), (4, 8), (5, 9), (6, 8), and (7, 9) in sequence. As will be described later, the hooking portion 22 is formed at each bending point. Similarly thereafter, the first wire is woven from the leftmost grid point (0, 8), which is identical to the rightmost grid point (8, 8), toward the rightmost grid point (8, 6). The first wire is woven from the leftmost grid point (0, 6), which is identical to the rightmost grid point (8, 6), toward the rightmost grid point (8, 4). The first wire is woven from the leftmost grid point (0, 4), which is identical to the rightmost grid point (8, 4), toward the rightmost grid point (8, 2). The first wire is woven from the leftmost grid point (0, 2), which is identical to the rightmost grid point (8, 2), toward a rightmost grid point (8, 0).

Note that no hooking portions 22 are formed at bending points (2, 0), (4, 0), and (6, 0) and the rightmost grid point (8, 0) of the stent 2 located at the upper end. Subsequently to the leftmost grid point (0, 0), which is identical to the rightmost grid point (8, 0), the first wire bends at the bending point (2, 2) and then is brought out from an uppermost grid point or an ending point (4, 0).

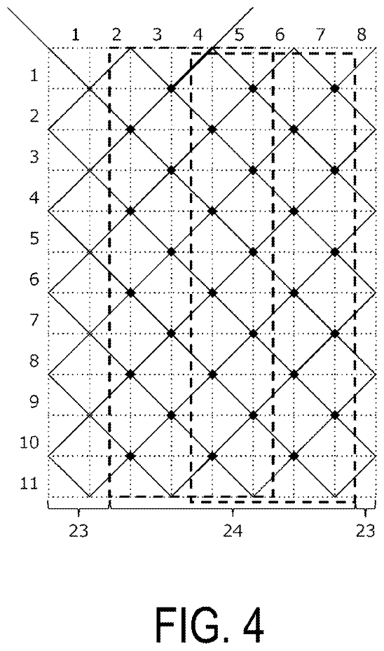

FIG. 4 schematically illustrates a result of weaving the first wire forming the stent 2 according to the first embodiment back and forth along the axial direction. This drawing is obtained by superimposing FIG. 3A representing the forward path and FIG. 3B representing the backward path on each other. The hooking portion 22 formed as a result of interlocking of the first wire is indicated as a rhomboid at each grid point. In the stent 2 (the first wire), a stretch assisting region 23 including a larger number of the crossing portions 21 than the hooking portions 22 and a contraction assisting region 24 including a larger number of the hooking portions 22 than the crossing portions 21 are separately disposed along the circumferential direction. Note that, in FIG. 4 illustrating the stent 2 deployed in the circumferential direction (horizontal direction), the stretch assisting region 23 is divided into left and right regions. However, in the actual stent 2 having a tubular shape, the one stretch assisting region 23 continuous in the circumferential direction is formed. That is, in the first embodiment of FIG. 4, the circumference of the stent 2 having a tubular shape is divided into the one stretch assisting region 23 and the one contraction assisting region 24.

In the stretch assisting region 23, at least two stretch assisting columns each of which includes a larger number of the crossing portions 21 than the hooking portions 22 in the axial direction (vertical direction) of the stent 2 are continuously disposed in the circumferential direction (horizontal direction) of the stent 2. In the example illustrated, two columns of the 0th (8th) column and the 1st column continuous in the circumferential direction are the stretch assisting columns. These stretch assisting columns include the crossing portions 21 only, and thus the number of the crossing portions 21 is larger than the number of the hooking portions 22. In the contraction assisting region 24, at least one or preferably a plurality of contraction assisting columns each of which includes a larger number of the hooking portions 22 than the crossing portions 21 in the axial direction (vertical direction) of the stent 2 are disposed. In the example illustrated, six columns of the 2nd column to the 7th column continuous in the circumferential direction are the contraction assisting columns. These contraction assisting columns include the hooking portions 22 only, and thus the number of the hooking portions 22 is larger than the number of the crossing portions 21. Note that, in the contraction assisting region 24, one or a plurality of separate stretch assisting columns that are not continuous in the circumferential direction and thus do not constitute the stretch assisting region 23 may be disposed. In this manner, the contraction assisting region 24 including the plurality of contraction assisting columns is formed in a region in the circumferential direction excluding the stretch assisting region 23 including the at least two continuously-disposed stretch assisting columns.

Two rectangular dashed line frames illustrated in FIG. 4 indicate two examples of a first equally-divided region obtained by equally dividing the stent 2 in the circumferential direction by a plane including the axis of the stent 2 having a tubular shape. The two examples of the first equally-divided region illustrated in the drawing do not include the stretch assisting columns that are continuous in the circumferential direction and constitute the stretch assisting region 23 (that is, include the contraction assisting region 24 only). Thus, a second equally-divided region (a region outside the dashed line frames) includes the stretch assisting columns that are continuous in the circumferential direction and constitute the stretch assisting region 23. In this case, a majority of the hooking portions 22 are provided in the first equally-divided region (region inside the dashed line frames). Note that, although the same description will be given in the subsequent drawings, illustration of the rectangular dashed line frames will be omitted.

The stretch assisting region 23 and the contraction assisting region 24 extend along the axial direction (vertical direction in FIG. 4) of the stent 2 having a tubular shape. The stretch assisting region 23 in which the crossing portion 21 is superior in numbers is easy to stretch and hard to contract along the axial direction of the stent 2 having a tubular shape. The contraction assisting region 24 in which the hooking portion 22 is superior in numbers is easy to contract and hard to stretch along the axial direction of the stent 2 having a tubular shape. For this reason, when the stent graft 1 is placed at a dilation target site having a curvature, the contraction assisting region 24 which is easy to contract is disposed on the inner side having a large curvature and the stretch assisting region 23 which is easy to stretch is disposed on the outer side having a small curvature, so that the stent graft 1 can be easily curved.

In the first embodiment of FIG. 4, all the hooking portions 22 are provided in the contraction assisting region 24. However, in order to produce a significant difference in stretchability between the stretch assisting region 23 and the contraction assisting region 24, it is enough to provide a majority of the hooking portions 22 in the contraction assisting region 24 (or provide less than half of the hooking portions 22 in the stretch assisting region 23). For example, 70% or more of the hooking portions 22 may be provided in the contraction assisting region 24, or 90% or more of the hooking portions 22 may be provided in the contraction assisting region 24.

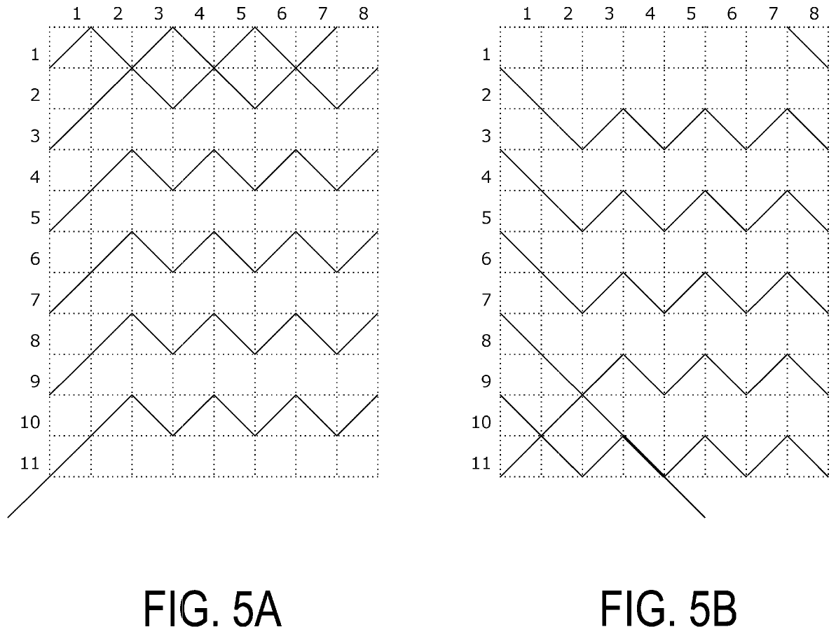

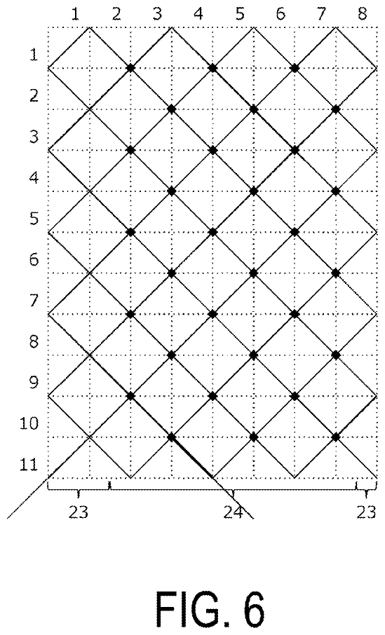

FIGS. 5A and 5B schematically illustrate a weaving method of a second wire forming the stent 2 according to the first embodiment. FIG. 5A illustrates a state in which the second wire forming the stent 2 is woven from the second end (the lower end) toward the first end (the upper end) in the axial direction. FIG. 5B illustrates a state in which, following FIG. 5A, the second wire is folded back at the first end (the upper end) and then woven from the first end (the upper end) toward the second end (the lower end) in the axial direction. In FIGS. 5A and 5B, and subsequent similar drawings, notations and a specific weaving method of the wire are the same as and/or similar to those in FIGS. 3A and 3B, and thus descriptions thereof will be omitted. The stent 2 according to the first embodiment is formed by combining the first wire in FIGS. 3A and 3B (or FIG. 4) and the second wire in FIGS. 5A and 5B (or FIG. 6). However, the first wire and the second wire are independent from each other and no hooking portion 22 is formed between the first wire and the second wire in the first embodiment. In other words, only the crossing portions 21 are formed between the first wire and the second wire.

FIG. 6 schematically illustrates a result of weaving the second wire forming the stent 2 according to the first embodiment back and forth along the axial direction. This drawing is obtained by superimposing FIG. 5A representing the forward path and FIG. 5B representing the backward path on each other. The hooking portion 22 formed as a result of interlocking of the second wire is indicated as a rhomboid at each grid point. In the stent 2 (the second wire), the stretch assisting region 23 including a larger number of the crossing portions 21 than the hooking portions 22 and the contraction assisting region 24 including a larger number of the hooking portions 22 than the crossing portions 21 are separately disposed along the circumferential direction. In the first embodiment of FIG. 6, the circumference of the stent 2 having a tubular shape is divided into the one stretch assisting region 23 and the one contraction assisting region 24.

In the stretch assisting region 23, at least two stretch assisting columns each of which includes a larger number of the crossing portions 21 than the hooking portions 22 in the axial direction (vertical direction) of the stent 2 are continuously disposed in the circumferential direction (horizontal direction) of the stent 2. In the example illustrated, two columns of the 0th (8th) column and the 1st column continuous in the circumferential direction are the stretch assisting columns. In the contraction assisting region 24, at least one or preferably a plurality of contraction assisting columns each of which includes a larger number of the hooking portions 22 than the crossing portions 21 in the axial direction (vertical direction) of the stent 2 are disposed. In the example illustrated, six columns of the 2nd column to the 7th column continuous in the circumferential direction are the contraction assisting columns. In this manner, the contraction assisting region 24 including the plurality of contraction assisting columns is formed in a region in the circumferential direction excluding the stretch assisting region 23 including the at least two continuously-disposed stretch assisting columns. In a manner similar to FIG. 4, when the stent 2 is equally divided in the circumferential direction by a plane including the axis of the stent 2 and a first equally-divided region includes the at least two continuously-disposed stretch assisting columns (that is, the stretch assisting region 23), a second equally-divided region (including the contraction assisting region 24 only) includes a majority of the hooking portions 22.

In the first embodiment of FIG. 6, all the hooking portions 22 are provided in the contraction assisting region 24. However, in order to produce a significant difference in stretchability between the stretch assisting region 23 and the contraction assisting region 24, it is enough to provide a majority of the hooking portions 22 in the contraction assisting region 24 (or provide less than half of the hooking portions 22 in the stretch assisting region 23). For example, 70% or more of the hooking portions 22 may be provided in the contraction assisting region 24, or 90% or more of the hooking portions 22 may be provided in the contraction assisting region 24.

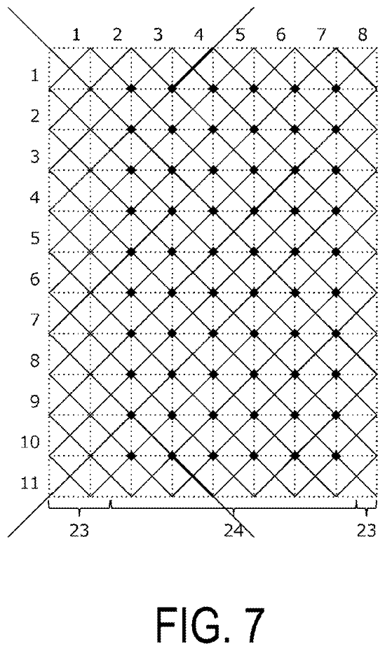

FIG. 7 schematically illustrates the stent 2 according to the first embodiment formed by combining the first wire and the second wire. This drawing is obtained by superimposing FIG. 4 representing the first wire and FIG. 6 representing the second wire on each other. In the stent 2, the stretch assisting region 23, which is an overlapping region between the first stretch assisting region 23 of the first wire (FIG. 4) and the second stretch assisting region 23 of the second wire (FIG. 6), and the contraction assisting region 24, which is an overlapping region between the first contraction assisting region 24 of the first wire (FIG. 4) and the second contraction assisting region 24 of the second wire (FIG. 6), are separately disposed along the circumferential direction. In the first embodiment of FIG. 7, the circumference of the stent 2 having a tubular shape is divided into the one stretch assisting region 23 and the one contraction assisting region 24.

In the stretch assisting region 23, at least three stretch assisting columns each of which includes a larger number of the crossing portions 21 than the hooking portions 22 in the axial direction (vertical direction) of the stent 2 are continuously disposed in the circumferential direction (horizontal direction) of the stent 2. As a result of combining the two wires, in addition to the eight columns of the 0th (8th) column to the 7th column constituting the grid points, additional eight columns including the crossing portions 21 between the first wire and the second wire are formed, each of which is located between corresponding adjacent two of the 0th (8th) to the 7th columns. That is, in the example of FIG. 7, a total of 16 columns are formed. Hereinafter, for example, a column formed between the 1st column and the 2nd column is referred to as “1.5th column” or the like. In the example illustrated, five columns of the 7.5th column, the 0th (8th) column, the 0.5th column, the 1st column, and the 1.5th column continuous in the circumferential direction are the stretch assisting columns. In the contraction assisting region 24, at least one or preferably a plurality of contraction assisting columns each of which includes a larger number of the hooking portions 22 than the crossing portions 21 in the axial direction (vertical direction) of the stent 2 are disposed. In the example illustrated, six columns of the 2nd column to the 7th column are the contraction assisting columns. The 2.5th column, the 3.5th column, the 4.5th column, the 5.5th column, and the 6.5th column formed between the respective contraction assisting columns by the crossing portions 21 between the first wire and the second wire are separate stretch assisting columns that are not continuous in the circumferential direction and thus do not constitute the stretch assisting region 23. In this manner, the contraction assisting region 24 including the plurality of contraction assisting columns is formed in a region in the circumferential direction excluding the stretch assisting region 23 including the at least three continuously-disposed stretch assisting columns. In a manner similar to FIG. 4, when the stent 2 is equally divided in the circumferential direction by a plane including the axis of the stent 2 and a first equally-divided region includes the at least three continuously-disposed stretch assisting columns (that is, the stretch assisting region 23), a second equally-divided region (including the contraction assisting region 24 only) includes a majority of the hooking portions 22.

The stretch assisting region 23 and the contraction assisting region 24 extend along the axial direction (vertical direction in FIG. 7) of the stent 2 having a tubular shape. The stretch assisting region 23 including a large number of the crossing portions 21 is easy to stretch and hard to contract along the axial direction of the stent 2 having a tubular shape. The contraction assisting region 24 including a large number of the hooking portions 22 is easy to contract and hard to stretch along the axial direction of the stent 2 having a tubular shape. For this reason, when the stent graft 1 is placed at a dilation target site having a curvature, the contraction assisting region 24 which is easy to contract is disposed on the inner side having a large curvature and the stretch assisting region 23 which is easy to stretch is disposed on the outer side having a small curvature, so that the stent graft 1 can be easily curved.

In the first embodiment of FIG. 7, all the hooking portions 22 are provided in the contraction assisting region 24. However, in order to produce a significant difference in stretchability between the stretch assisting region 23 and the contraction assisting region 24, it is enough to provide a majority of the hooking portions 22 in the contraction assisting region 24 (or provide less than half of the hooking portions 22 in the stretch assisting region 23). For example, 70% or more of the hooking portions 22 may be provided in the contraction assisting region 24, or 90% or more of the hooking portions 22 may be provided in the contraction assisting region 24.

In the first embodiment of FIG. 7, the first stretch assisting region 23 of the first wire and the second stretch assisting region 23 of the second wire completely coincide with each other, and the first contraction assisting region 24 of the first wire and the second contraction assisting region 24 of the second wire completely coincide with each other. However, it is enough that the first stretch assisting region 23 and the second stretch assisting region 23 overlap with each other and the first contraction assisting region 24 and the second contraction assisting region 24 overlap with each other.

As described above, in the first embodiment, no hooking portion 22 is formed between the first wire and the second wire, and only the crossing portions 21 are formed between the first wire and the second wire. Thus, the first wire and the second wire also form the crossing portions 21 in the overlapping region between the first contraction assisting region 24 and the second contraction assisting region 24. As a result, in the contraction assisting region 24 obtained by combining the first wire and the second wire, substantially equal numbers of the crossing portions 21 and the hooking portions 22 are formed. However, as illustrated in FIG. 4 (the first wire) and FIG. 6 (the second wire), when a focus is placed on each wire, a larger number of the hooking portions 22 than the crossing portions 21 are formed in the contraction assisting region 24.

Where X is the number of positions in the circumferential direction at which each wire can form the crossing portion 21 or the hooking portion 22 (the number of intermediate points, such as the 0.5th column, the 1.5th column, and 2.5th column, between integer number columns, or the grid points), Y is the number of positions in the axial direction at which each wire can form the crossing portion 21 or the hooking portion 22 (the number of intermediate points, such as the 0.5th row, the 1.5th row, and 2.5th row, between integer number rows, or the grid points), and Z is the number of times each wire is woven back and forth in the axial direction, the number of the hooking portions 22 is (X−Z)(Y−1), and the number of the crossing portions 21 is XY+(Y−1)Z. In the first embodiment of FIG. 7, X=8, Y=11, and Z=2 (each of the first wire and the second wire is woven back and forth once), and thus the number of the hooking portions 22 is (8 −2)(11 −1)=60, and the number of the crossing portions 21 is 8×11+(11 −1)×2=108. Additionally, in the first embodiment described above, the number X of the positions (grid points) in the circumferential direction at which each wire can form the crossing portion 21 or the hooking portion 22 is an even number (8), and the one stretch assisting region 23 and the one contraction assisting region 24 are disposed along the circumferential direction.

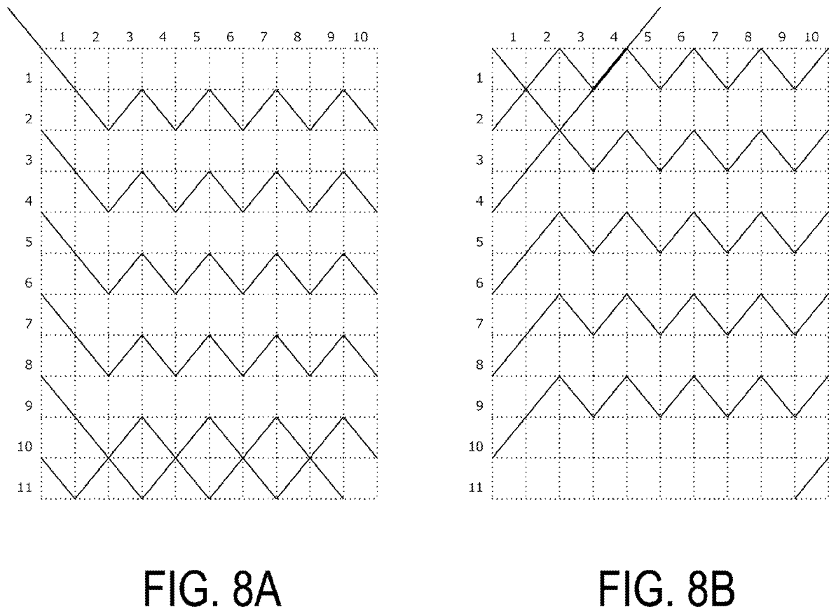

FIGS. 8A and 8B schematically illustrate a weaving method of the first wire forming the stent 2 according to a second embodiment. In the present embodiment, the total number X of the grid points in the circumferential direction is 10, and the total number Y of the grid points in the axial direction is 11. FIG. 8A illustrates a state in which the first wire forming the stent 2 is woven from a first end (the upper end) toward a second end (the lower end) in the axial direction. FIG. 8B illustrates a state in which, following FIG. 8A, the first wire is folded back at the second end (the lower end) and then woven from the second end (the lower end) toward the first end (the upper end) in the axial direction.

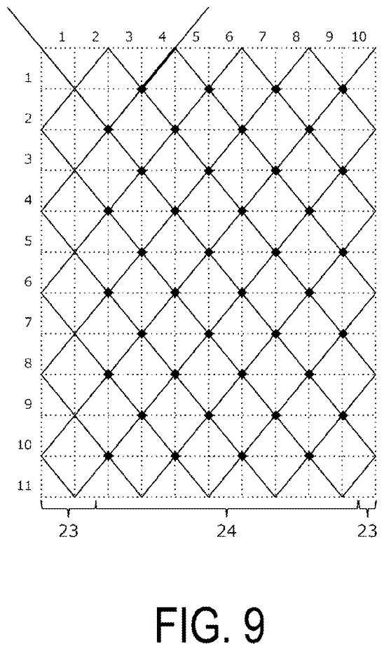

FIG. 9 schematically illustrates a result of weaving the first wire forming the stent 2 according to the second embodiment back and forth along the axial direction. This drawing is obtained by superimposing FIG. 8A representing the forward path and FIG. 8B representing the backward path on each other. In the stent 2 (the first wire), the stretch assisting region 23 including a larger number of the crossing portions 21 than the hooking portions 22 and the contraction assisting region 24 including a larger number of the hooking portions 22 than the crossing portions 21 are separately disposed along the circumferential direction. In the second embodiment of FIG. 9, the circumference of the stent 2 having a tubular shape is divided into the one stretch assisting region 23 and the one contraction assisting region 24.

In the stretch assisting region 23, at least two stretch assisting columns each of which includes a larger number of the crossing portions 21 than the hooking portions 22 in the axial direction (vertical direction) of the stent 2 are continuously disposed in the circumferential direction (horizontal direction) of the stent 2. In the example illustrated, two columns of the 0th (10th) column and the 1st column continuous in the circumferential direction are the stretch assisting columns. In the contraction assisting region 24, at least one or preferably a plurality of contraction assisting columns each of which includes a larger number of the hooking portions 22 than the crossing portions 21 in the axial direction (vertical direction) of the stent 2 are disposed. In the example illustrated, eight columns of the 2nd column to the 9th column continuous in the circumferential direction are the contraction assisting columns. In this manner, the contraction assisting region 24 including the plurality of contraction assisting columns is formed in a region in the circumferential direction excluding the stretch assisting region 23 including the at least two continuously-disposed stretch assisting columns. In a manner similar to FIG. 4, when the stent 2 is equally divided in the circumferential direction by a plane including the axis of the stent 2 and a first equally-divided region includes the at least two continuously-disposed stretch assisting columns (that is, the stretch assisting region 23), a second equally-divided region (including the contraction assisting region 24 only) includes a majority of the hooking portions 22.

The stretch assisting region 23 and the contraction assisting region 24 extend along the axial direction (vertical direction in FIG. 9) of the stent 2 having a tubular shape. The stretch assisting region 23 in which the crossing portion 21 is superior in numbers is easy to stretch and hard to contract along the axial direction of the stent 2 having a tubular shape. The contraction assisting region 24 in which the hooking portion 22 is superior in numbers is easy to contract and hard to stretch along the axial direction of the stent 2 having a tubular shape. For this reason, when the stent graft 1 is placed at a dilation target site having a curvature, the contraction assisting region 24 which is easy to contract is disposed on the inner side having a large curvature and the stretch assisting region 23 which is easy to stretch is disposed on the outer side having a small curvature, so that the stent graft 1 can be easily curved.

In the second embodiment of FIG. 9, all the hooking portions 22 are provided in the contraction assisting region 24. However, in order to produce a significant difference in stretchability between the stretch assisting region 23 and the contraction assisting region 24, it is enough to provide a majority of the hooking portions 22 in the contraction assisting region 24 (or provide less than half of the hooking portions 22 in the stretch assisting region 23). For example, 70% or more of the hooking portions 22 may be provided in the contraction assisting region 24, or 90% or more of the hooking portions 22 may be provided in the contraction assisting region 24.

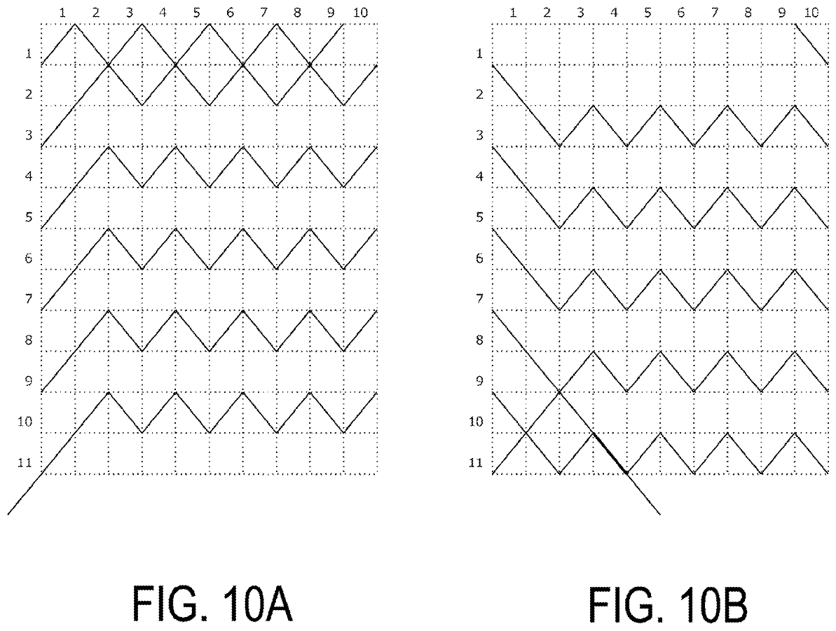

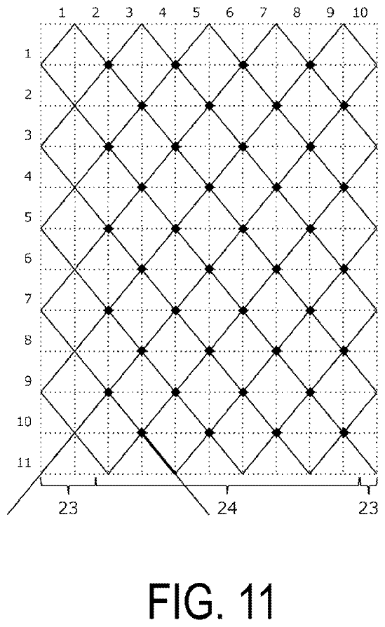

FIGS. 10A and 10B schematically illustrate a weaving method of the second wire forming the stent 2 according to the second embodiment. FIG. 10A illustrates a state in which the second wire forming the stent 2 is woven from the second end (the lower end) toward the first end (the upper end) in the axial direction. FIG. 10B illustrates a state in which, following FIG. 10A, the second wire is folded back at the first end (the upper end) and then woven from the first end (the upper end) toward the second end (the lower end) in the axial direction. The stent 2 according to the second embodiment is formed by combining the first wire in FIGS. 8A and 8B (or FIG. 9) and the second wire in FIGS. 10A and 10B (or FIG. 11). However, the first wire and the second wire are independent from each other and no hooking portion 22 is formed between the first wire and the second wire in the second embodiment. In other words, only the crossing portions 21 are formed between the first wire and the second wire.

FIG. 11 schematically illustrates a result of weaving the second wire forming the stent 2 according to the second embodiment back and forth along the axial direction. This drawing is obtained by superimposing FIG. 10A representing the forward path and FIG. 10B representing the backward path on each other. In the stent 2 (the second wire), the stretch assisting region 23 including a larger number of the crossing portions 21 than the hooking portions 22 and the contraction assisting region 24 including a larger number of the hooking portions 22 than the crossing portions 21 are separately disposed along the circumferential direction. In the second embodiment of FIG. 11, the circumference of the stent 2 having a tubular shape is divided into the one stretch assisting region 23 and the one contraction assisting region 24.

In the stretch assisting region 23, at least two stretch assisting columns each of which includes a larger number of the crossing portions 21 than the hooking portions 22 in the axial direction (vertical direction) of the stent 2 are continuously disposed in the circumferential direction (horizontal direction) of the stent 2. In the example illustrated, two columns of the 0th (10th) column and the 1st column continuous in the circumferential direction are the stretch assisting columns. In the contraction assisting region 24, at least one or preferably a plurality of contraction assisting columns each of which includes a larger number of the hooking portions 22 than the crossing portions 21 in the axial direction (vertical direction) of the stent 2 are disposed. In the example illustrated, eight columns of the 2nd column to the 9th column continuous in the circumferential direction are the contraction assisting columns. In this manner, the contraction assisting region 24 including the plurality of contraction assisting columns is formed in a region in the circumferential direction excluding the stretch assisting region 23 including the at least two continuously-disposed stretch assisting columns. In a manner similar to FIG. 4, when the stent 2 is equally divided in the circumferential direction by a plane including the axis of the stent 2 and a first equally-divided region includes the at least two continuously-disposed stretch assisting columns (that is, the stretch assisting region 23), a second equally-divided region (including the contraction assisting region 24 only) includes a majority of the hooking portions 22.

In the second embodiment of FIG. 11, all the hooking portions 22 are provided in the contraction assisting region 24. However, in order to produce a significant difference in stretchability between the stretch assisting region 23 and the contraction assisting region 24, it is enough to provide a majority of the hooking portions 22 in the contraction assisting region 24 (or provide less than half of the hooking portions 22 in the stretch assisting region 23). For example, 70% or more of the hooking portions 22 may be provided in the contraction assisting region 24, or 90% or more of the hooking portions 22 may be provided in the contraction assisting region 24.

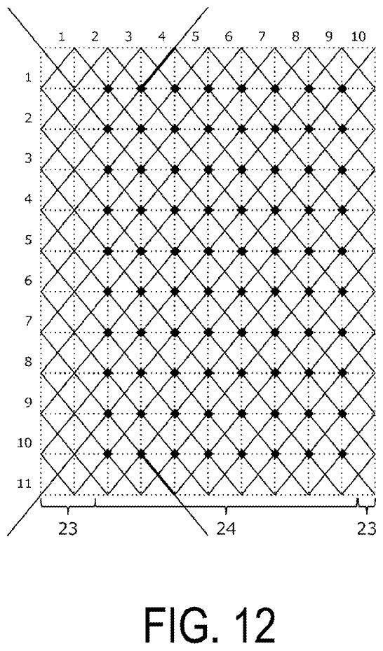

FIG. 12 schematically illustrates the stent 2 according to the second embodiment formed by combining the first wire and the second wire. This drawing is obtained by superimposing FIG. 9 representing the first wire and FIG. 11 representing the second wire on each other. In the stent 2, the stretch assisting region 23, which is an overlapping region between the first stretch assisting region 23 of the first wire (FIG. 9) and the second stretch assisting region 23 of the second wire (FIG. 11), and the contraction assisting region 24, which is an overlapping region between the first contraction assisting region 24 of the first wire (FIG. 9) and the second contraction assisting region 24 of the second wire (FIG. 11), are separately disposed along the circumferential direction. In the second embodiment of FIG. 12, the circumference of the stent 2 having a tubular shape is divided into the one stretch assisting region 23 and the one contraction assisting region 24.

In the stretch assisting region 23, at least three stretch assisting columns each of which includes a larger number of the crossing portions 21 than the hooking portions 22 in the axial direction (vertical direction) of the stent 2 are continuously disposed in the circumferential direction (horizontal direction) of the stent 2. As a result of combining the two wires, in addition to the ten columns of the 0th (10th) column to the 9th column constituting the grid points, additional 10 columns including the crossing portions 21 between the first wire and the second wire are formed, each of which is located between corresponding adjacent two of the 0th (10th) to the 9th columns. That is, in the example of FIG. 12, a total of 20 columns are formed. In the example illustrated, five columns of the 9.5th column, the 0th (10th) column, the 0.5th column, the 1st column, and the 1.5th column continuous in the circumferential direction are the stretch assisting columns. In the contraction assisting region 24, at least one or preferably a plurality of contraction assisting columns each of which includes a larger number of the hooking portions 22 than the crossing portions 21 in the axial direction (vertical direction) of the stent 2 are disposed. In the example illustrated, eight columns of the 2nd column to the 9th column are the contraction assisting columns. The 2.5th column, the 3.5th column, the 4.5th column, the 5.5th column, the 6.5th column, the 7.5th column, and the 8.5th column formed between the respective contraction assisting columns by the crossing portions 21 between the first wire and the second wire are separate stretch assisting columns that are not continuous in the circumferential direction and thus do not constitute the stretch assisting region 23. In this manner, the contraction assisting region 24 including the plurality of contraction assisting columns is formed in a region in the circumferential direction excluding the stretch assisting region 23 including the at least three continuously-disposed stretch assisting columns. In a manner similar to FIG. 4, when the stent 2 is equally divided in the circumferential direction by a plane including the axis of the stent 2 and a first equally-divided region includes the at least three continuously-disposed stretch assisting columns (that is, the stretch assisting region 23), a second equally-divided region (including the contraction assisting region 24 only) includes a majority of the hooking portions 22.

The stretch assisting region 23 and the contraction assisting region 24 extend along the axial direction (vertical direction in FIG. 12) of the stent 2 having a tubular shape. The stretch assisting region 23 including a large number of the crossing portions 21 is easy to stretch and hard to contract along the axial direction of the stent 2 having a tubular shape. The contraction assisting region 24 including a large number of the hooking portions 22 is easy to contract and hard to stretch along the axial direction of the stent 2 having a tubular shape. For this reason, when the stent graft 1 is placed at a dilation target site having a curvature, the contraction assisting region 24 which is easy to contract is disposed on the inner side having a large curvature and the stretch assisting region 23 which is easy to stretch is disposed on the outer side having a small curvature, so that the stent graft 1 can be easily curved.

In the second embodiment of FIG. 12, all the hooking portions 22 are provided in the contraction assisting region 24. However, in order to produce a significant difference in stretchability between the stretch assisting region 23 and the contraction assisting region 24, it is enough to provide a majority of the hooking portions 22 in the contraction assisting region 24 (or provide less than half of the hooking portions 22 in the stretch assisting region 23). For example, 70% or more of the hooking portions 22 may be provided in the contraction assisting region 24, or 90% or more of the hooking portions 22 may be provided in the contraction assisting region 24.

In the second embodiment of FIG. 12, the first stretch assisting region 23 of the first wire and the second stretch assisting region 23 of the second wire completely coincide with each other, and the first contraction assisting region 24 of the first wire and the second contraction assisting region 24 of the second wire completely coincide with each other. However, it is enough that the first stretch assisting region 23 and the second stretch assisting region 23 overlap with each other and the first contraction assisting region 24 and the second contraction assisting region 24 overlap with each other.

As described above, in the second embodiment, no hooking portion 22 is formed between the first wire and the second wire, and only the crossing portions 21 are formed between the first wire and the second wire. Thus, the first wire and the second wire also form the crossing portions 21 in the overlapping region between the first contraction assisting region 24 and the second contraction assisting region 24. As a result, in the contraction assisting region 24 obtained by combining the first wire and the second wire, substantially equal numbers of the crossing portions 21 and the hooking portions 22 are formed. However, as illustrated in FIG. 9 (the first wire) and FIG. 11 (the second wire), when a focus is placed on each wire, a larger number of the hooking portions 22 than the crossing portions 21 are formed in the contraction assisting region 24.

As described in the first embodiment, the number of the hooking portions 22 is (X−Z)(Y−1), and the number of the crossing portions 21 is XY+(Y−1)Z. In the second embodiment of FIG. 12, X=10, Y=11, and Z=2 (each of the first wire and the second wire is woven back and forth once), and thus the number of the hooking portions 22 is (10 −2)(11 −1)=80, and the number of the crossing portions 21 is 10×11+(11 −1)×2=130. Additionally, in the second embodiment described above, the number X of the positions (grid points) in the circumferential direction at which each wire can form the crossing portion 21 or the hooking portion 22 is an even number (10), and the one stretch assisting region 23 and the one contraction assisting region 24 are disposed along the circumferential direction.

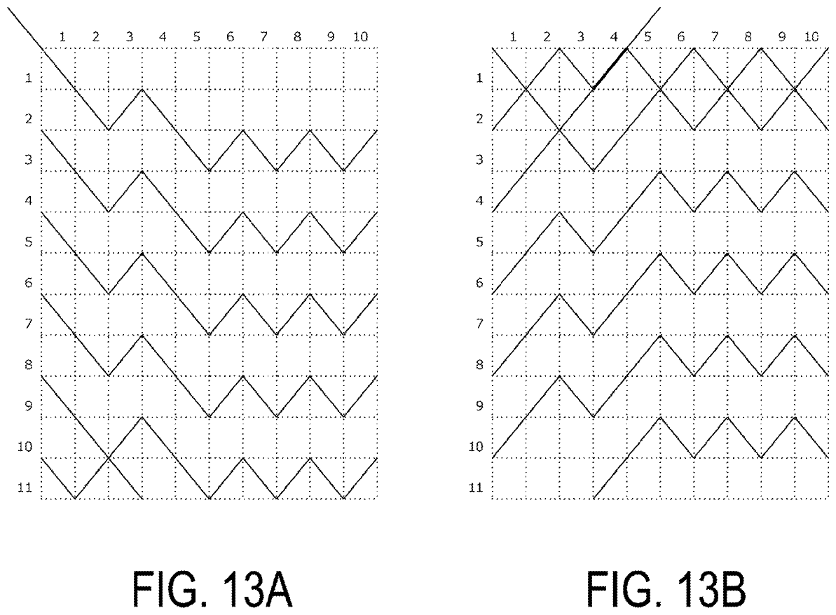

FIGS. 13A and 13B schematically illustrate a weaving method of the first wire forming the stent 2 according to a third embodiment. In the present embodiment, the total number X of the grid points in the circumferential direction is 10, and the total number Y of the grid points in the axial direction is 11. FIG. 13A illustrates a state in which the first wire forming the stent 2 is woven from a first end (the upper end) toward a second end (the lower end) in the axial direction. FIG. 13B illustrates a state in which, following FIG. 13A, the first wire is folded back at the second end (the lower end) and then woven from the second end (the lower end) toward the first end (the upper end) in the axial direction.

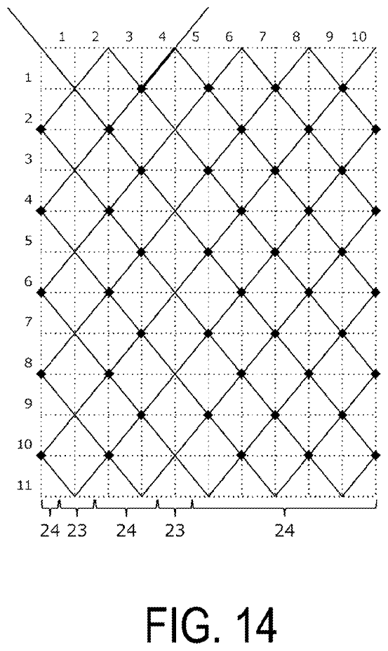

FIG. 14 schematically illustrates a result of weaving the first wire forming the stent 2 according to the third embodiment back and forth along the axial direction. This drawing is obtained by superimposing FIG. 13A representing the forward path and FIG. 13B representing the backward path on each other. In the stent 2 (the first wire), the stretch assisting regions 23 each including a larger number of the crossing portions 21 than the hooking portions 22 and the contraction assisting regions 24 each including a larger number of the hooking portions 22 than the crossing portions 21 are separately disposed along the circumferential direction. In the third embodiment of FIG. 14, the circumference of the stent 2 having a tubular shape is divided into the two stretch assisting regions 23 and the two contraction assisting regions 24.

The stretch assisting regions 23 and the contraction assisting regions 24 extend along the axial direction (vertical direction in FIG. 14) of the stent 2 having a tubular shape. Each stretch assisting region 23 in which the crossing portion 21 is superior in numbers is easy to stretch and hard to contract along the axial direction of the stent 2 having a tubular shape. Each contraction assisting region 24 in which the hooking portion 22 is superior in numbers is easy to contract and hard to stretch along the axial direction of the stent 2 having a tubular shape. For this reason, when the stent graft 1 is placed at a dilation target site having a curvature, the contraction assisting regions 24 which are easy to contract are disposed on the inner side having a large curvature and the stretch assisting regions 23 which are easy to stretch are disposed on the outer side having a small curvature, so that the stent graft 1 can be easily curved. In particular, the stretch assisting region 23 (and the contraction assisting region 24) formed as one region in the first embodiment and the second embodiment is divided into a plurality of regions and distributed along the circumferential direction in the present embodiment. Thus, even when the stent graft 1 turns around the axial direction while being placed at a dilation target site having a curvature, the stent graft 1 can be stably curved.

In the third embodiment of FIG. 14, all the hooking portions 22 are provided in the contraction assisting regions 24. However, in order to produce a significant difference in stretchability between the stretch assisting regions 23 and the contraction assisting regions 24, it is enough to provide a majority of the hooking portions 22 in the contraction assisting regions 24 (or provide less than half of the hooking portions 22 in the stretch assisting regions 23). For example, 70% or more of the hooking portions 22 may be provided in the contraction assisting regions 24, or 90% or more of the hooking portions 22 may be provided in the contraction assisting regions 24.

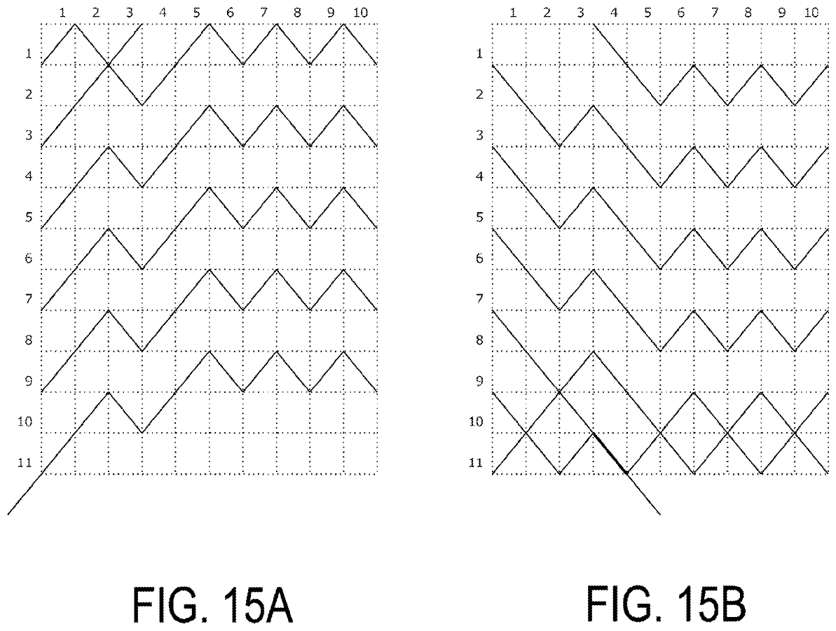

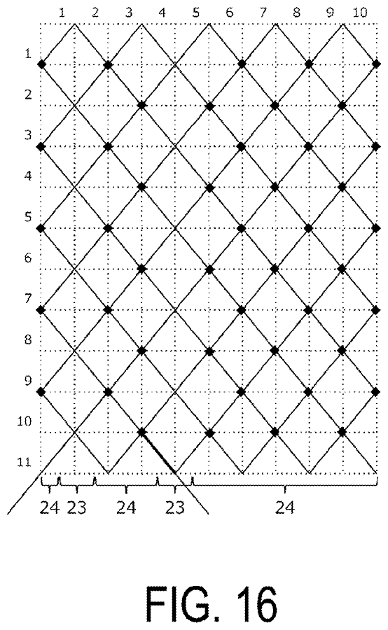

FIGS. 15A and 15B schematically illustrate a weaving method of the second wire forming the stent 2 according to the third embodiment. FIG. 15A illustrates a state in which the second wire forming the stent 2 is woven from the second end (the lower end) toward the first end (the upper end) in the axial direction. FIG. 15B illustrates a state in which, following FIG. 15A, the second wire is folded back at the first end (the upper end) and then woven from the first end (the upper end) toward the second end (the lower end) in the axial direction. The stent 2 according to the third embodiment is formed by combining the first wire in FIGS. 13A and 13B (or FIG. 14) and the second wire in FIGS. 15A and 15B (or FIG. 16). However, the first wire and the second wire are independent from each other and no hooking portion 22 is formed between the first wire and the second wire in the third embodiment. In other words, only the crossing portions 21 are formed between the first wire and the second wire.

FIG. 16 schematically illustrates a result of weaving the second wire forming the stent 2 according to the third embodiment back and forth along the axial direction. This drawing is obtained by superimposing FIG. 15A representing the forward path and FIG. 15B representing the backward path on each other. In the stent 2 (the second wire), the stretch assisting regions 23 each including a larger number of the crossing portions 21 than the hooking portions 22 and the contraction assisting regions 24 each including a larger number of the hooking portions 22 than the crossing portions 21 are separately disposed along the circumferential direction. In the third embodiment of FIG. 16, the circumference of the stent 2 having a tubular shape is divided into the two stretch assisting regions 23 and the two contraction assisting regions 24.

In the third embodiment of FIG. 16, all the hooking portions 22 are provided in the contraction assisting regions 24. However, in order to produce a significant difference in stretchability between the stretch assisting regions 23 and the contraction assisting regions 24, it is enough to provide a majority of the hooking portions 22 in the contraction assisting regions 24 (or provide less than half of the hooking portions 22 in the stretch assisting regions 23). For example, 70% or more of the hooking portions 22 may be provided in the contraction assisting regions 24, or 90% or more of the hooking portions 22 may be provided in the contraction assisting regions 24.

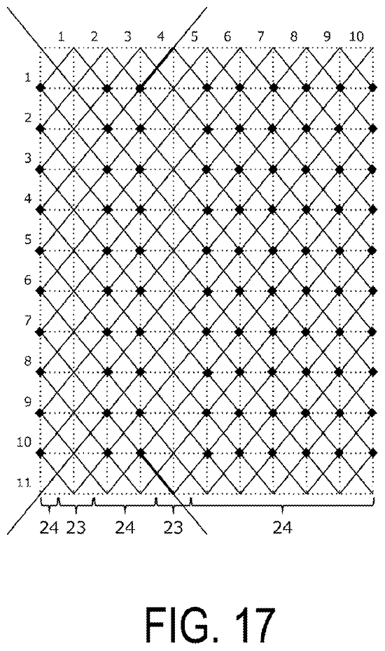

FIG. 17 schematically illustrates the stent 2 according to the third embodiment formed by combining the first wire and the second wire. This drawing is obtained by superimposing FIG. 14 representing the first wire and FIG. 16 representing the second wire on each other. In the stent 2, the stretch assisting regions 23, which are overlapping regions between the first stretch assisting regions 23 of the first wire (FIG. 14) and the second stretch assisting regions 23 of the second wire (FIG. 16), and the contraction assisting regions 24, which are overlapping regions between the first contraction assisting regions 24 of the first wire (FIG. 14) and the second contraction assisting regions 24 of the second wire (FIG. 16), are separately disposed along the circumferential direction. In the third embodiment of FIG. 17, the circumference of the stent 2 having a tubular shape is divided into the two stretch assisting regions 23 and the two contraction assisting regions 24. As described above, when the plurality of stretch assisting regions 23, each of which includes at least two continuously-disposed stretch assisting columns, are disposed separately from each other in the circumferential direction, a total of at least four stretch assisting columns are present in the plurality of stretch assisting regions 23, and a total of at least two contraction assisting columns are present in the plurality of contraction assisting regions 24 between the stretch assisting regions 23. Accordingly, the number of positions in the circumferential direction at which each wire can form the intersecting portion 21 or the interlocking portion 22 is 6 or more.

In each stretch assisting region 23, at least three stretch assisting columns each of which includes a larger number of the crossing portions 21 than the hooking portions 22 in the axial direction (vertical direction) of the stent 2 are continuously disposed in the circumferential direction (horizontal direction) of the stent 2. As a result of combining the two wires, in addition to the ten columns of the 0th (10th) column to the 9th column constituting the grid points, additional 10 columns including the crossing portions 21 between the first wire and the second wire are formed, each of which is located between corresponding adjacent two of the 0th (10th) to the 9th columns. That is, in the example of FIG. 17, a total of 20 columns are formed. In the example illustrated, three columns of the 0.5th column, the 1st column, and the 1.5th column continuous in the circumferential direction, and three columns of the 3.5th column, the 4th column, and the 4.5th column continuous in the circumferential direction are the stretch assisting columns. In this manner, the plurality of stretch assisting regions 23, each of which includes the at least three continuously-disposed stretch assisting columns, are disposed separately from each other in the circumferential direction. In each contraction assisting region 24, at least one or preferably a plurality of contraction assisting columns each of which includes a larger number of the hooking portions 22 than the crossing portions 21 in the axial direction (vertical direction) of the stent 2 are disposed. In the example illustrated, eight columns of the 2nd column, the 3rd column, and the 5th column to the 10th column are the contraction assisting columns. The 2.5th column, the 5.5th column, the 6.5th column, the 7.5th column, the 8.5th column, and the 9.5th column formed between adjacent contraction assisting columns by the crossing portions 21 between the first wire and the second wire are separate stretch assisting columns that are not continuous in the circumferential direction and thus do not constitute the stretch assisting regions 23. In this manner, the contraction assisting regions 24 each including the plurality of contraction assisting columns are formed in regions in the circumferential direction excluding the stretch assisting regions 23 each including the at least three continuously-disposed stretch assisting columns. In a manner similar to FIG. 4, when the stent 2 is equally divided in the circumferential direction by a plane including the axis of the stent 2 and a first equally-divided region includes the at least three continuously-disposed stretch assisting columns (that is, the stretch assisting region 23), a second equally-divided region (including the contraction assisting region 24 only) includes a majority of the hooking portions 22.

The stretch assisting regions 23 and the contraction assisting regions 24 extend along the axial direction (vertical direction in FIG. 17) of the stent 2 having a tubular shape. Each stretch assisting region 23 including a large number of the crossing portions 21 is easy to stretch and hard to contract along the axial direction of the stent 2 having a tubular shape. Each contraction assisting region 24 including a large number of the hooking portions 22 is easy to contract and hard to stretch along the axial direction of the stent 2 having a tubular shape. For this reason, when the stent graft 1 is placed at a dilation target site having a curvature, the contraction assisting regions 24 which are easy to contract are disposed on the inner side having a large curvature and the stretch assisting regions 23 which are easy to stretch are disposed on the outer side having a small curvature, so that the stent graft 1 can be easily curved. In particular, the stretch assisting region 23 (and the contraction assisting region 24) formed as one region in the first embodiment and the second embodiment is divided into a plurality of regions and distributed along the circumferential direction in the present embodiment. Thus, even when the stent graft 1 turns around the axial direction while being placed at a dilation target site having a curvature, the stent graft 1 can be stably curved.

In the third embodiment of FIG. 17, all the hooking portions 22 are provided in the contraction assisting regions 24. However, in order to produce a significant difference in stretchability between the stretch assisting regions 23 and the contraction assisting regions 24, it is enough to provide a majority of the hooking portions 22 in the contraction assisting regions 24 (or provide less than half of the hooking portions 22 in the stretch assisting regions 23). For example, 70% or more of the hooking portions 22 may be provided in the contraction assisting regions 24, or 90% or more of the hooking portions 22 may be provided in the contraction assisting regions 24.

In the third embodiment of FIG. 17, the first stretch assisting regions 23 of the first wire and the second stretch assisting regions 23 of the second wire completely coincide with each other, and the first contraction assisting regions 24 of the first wire and the second contraction assisting regions 24 of the second wire completely coincide with each other. However, it is enough that the first stretch assisting regions 23 and the second stretch assisting regions 23 overlap with each other and the first contraction assisting regions 24 and the second contraction assisting regions 24 overlap with each other.

As described above, in the third embodiment, no hooking portion 22 is formed between the first wire and the second wire, and only the crossing portions 21 are formed between the first wire and the second wire. Thus, the first wire and the second wire also form the crossing portions 21 in the overlapping regions between the first contraction assisting regions 24 and the second contraction assisting regions 24. As a result, in the contraction assisting regions 24 obtained by combining the first wire and the second wire, substantially equal numbers of the crossing portions 21 and the hooking portions 22 are formed. However, as illustrated in FIG. 14 (the first wire) and FIG. 16 (the second wire), when a focus is placed on each wire, a larger number of the hooking portions 22 than the crossing portions 21 are formed in the contraction assisting regions 24.

As described in the first embodiment, the number of the hooking portions 22 is (X−Z)(Y−1), and the number of the crossing portions 21 is XY+(Y−1)Z. In the third embodiment of FIG. 17, X=10, Y=11, and Z=2 (each of the first wire and the second wire is woven back and forth once), and thus the number of the hooking portions 22 is (10 −2)(11 −1)=80, and the number of the crossing portions 21 is 10×11+(11 −1)×2=130. Additionally, in the third embodiment described above, the number X of the positions (grid points) in the circumferential direction at which each wire can form the crossing portion 21 or the hooking portion 22 is an even number (10), and the plurality of stretch assisting regions 23 are disposed separately from each other along the circumferential direction, and the plurality of contraction assisting regions 24 are disposed separately from each other along the circumferential direction.

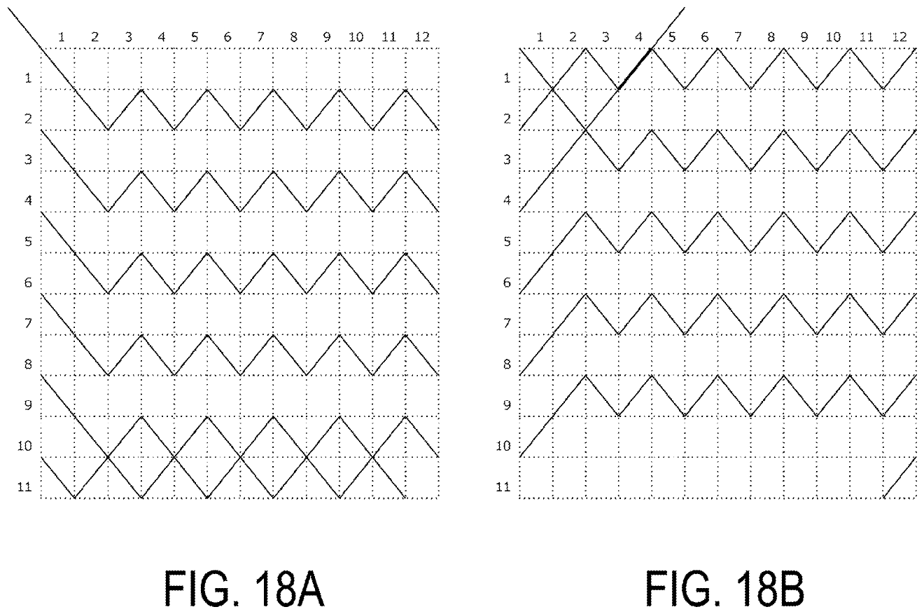

FIGS. 18A and 18B schematically illustrate a weaving method of the first wire forming the stent 2 according to a fourth embodiment. In the present embodiment, the total number X of the grid points in the circumferential direction is 12, and the total number Y of the grid points in the axial direction is 11. FIG. 18A illustrates a state in which the first wire forming the stent 2 is woven from a first end (the upper end) toward a second end (the lower end) in the axial direction. FIG. 18B illustrates a state in which, following FIG. 18A, the first wire is folded back at the second end (the lower end) and then woven from the second end (the lower end) toward the first end (the upper end) in the axial direction.

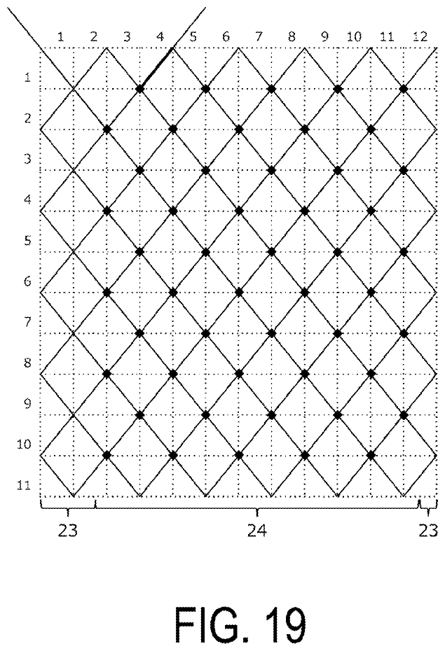

FIG. 19 schematically illustrates a result of weaving the first wire forming the stent 2 according to the fourth embodiment back and forth along the axial direction. This drawing is obtained by superimposing FIG. 18A representing the forward path and FIG. 18B representing the backward path on each other. In the stent 2 (the first wire), the stretch assisting region 23 including a larger number of the crossing portions 21 than the hooking portions 22 and the contraction assisting region 24 including a larger number of the hooking portions 22 than the crossing portions 21 are separately disposed along the circumferential direction. In the fourth embodiment of FIG. 19, the circumference of the stent 2 having a tubular shape is divided into the one stretch assisting region 23 and the one contraction assisting region 24.

In the stretch assisting region 23, at least two stretch assisting columns each of which includes a larger number of the crossing portions 21 than the hooking portions 22 in the axial direction (vertical direction) of the stent 2 are continuously disposed in the circumferential direction (horizontal direction) of the stent 2. In the example illustrated, two columns of the 0th (12th) column and the 1st column continuous in the circumferential direction are the stretch assisting columns. In the contraction assisting region 24, at least one or preferably a plurality of contraction assisting columns each of which includes a larger number of the hooking portions 22 than the crossing portions 21 in the axial direction (vertical direction) of the stent 2 are disposed. In the example illustrated, ten columns of the 2nd column to the 11th column continuous in the circumferential direction are the contraction assisting columns. In this manner, the contraction assisting region 24 including the plurality of contraction assisting columns is formed in a region in the circumferential direction excluding the stretch assisting region 23 including the at least two continuously-disposed stretch assisting columns. In a manner similar to FIG. 4, when the stent 2 is equally divided in the circumferential direction by a plane including the axis of the stent 2 and a first equally-divided region includes the at least two continuously-disposed stretch assisting columns (that is, the stretch assisting region 23), a second equally-divided region (including the contraction assisting region 24 only) includes a majority of the hooking portions 22.

The stretch assisting region 23 and the contraction assisting region 24 extend along the axial direction (vertical direction in FIG. 19) of the stent 2 having a tubular shape. The stretch assisting region 23 in which the crossing portion 21 is superior in numbers is easy to stretch and hard to contract along the axial direction of the stent 2 having a tubular shape. The contraction assisting region 24 in which the hooking portion 22 is superior in numbers is easy to contract and hard to stretch along the axial direction of the stent 2 having a tubular shape. For this reason, when the stent graft 1 is placed at a dilation target site having a curvature, the contraction assisting region 24 which is easy to contract is disposed on the inner side having a large curvature and the stretch assisting region 23 which is easy to stretch is disposed on the outer side having a small curvature, so that the stent graft 1 can be easily curved.

In the fourth embodiment of FIG. 19, all the hooking portions 22 are provided in the contraction assisting region 24. However, in order to produce a significant difference in stretchability between the stretch assisting region 23 and the contraction assisting region 24, it is enough to provide a majority of the hooking portions 22 in the contraction assisting region 24 (or provide less than half of the hooking portions 22 in the stretch assisting region 23). For example, 70% or more of the hooking portions 22 may be provided in the contraction assisting region 24, or 90% or more of the hooking portions 22 may be provided in the contraction assisting region 24.

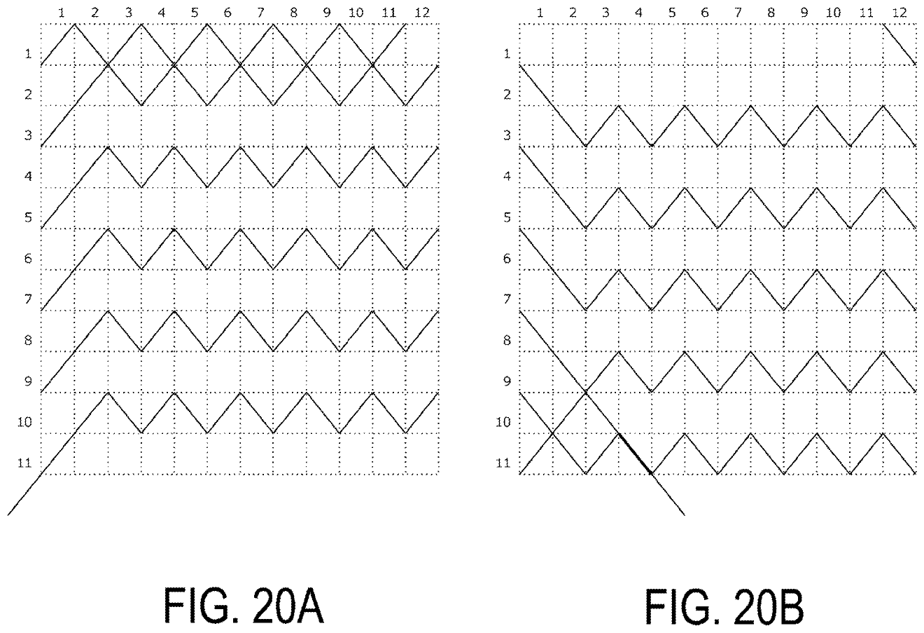

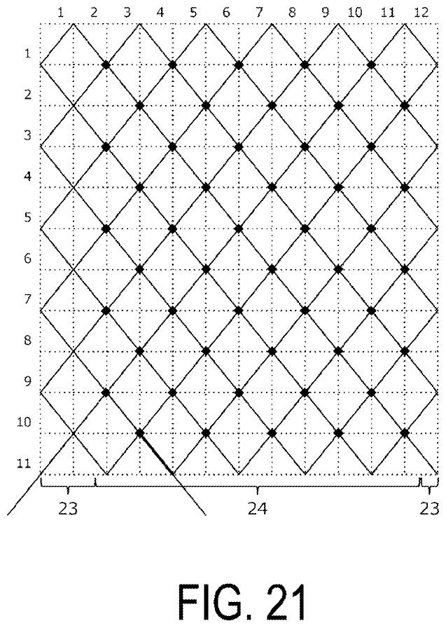

FIGS. 20A and 20B schematically illustrate a weaving method of the second wire forming the stent 2 according to the fourth embodiment. FIG. 20A illustrates a state in which the second wire forming the stent 2 is woven from the second end (the lower end) toward the first end (the upper end) in the axial direction. FIG. 20B illustrates a state in which, following FIG. 20A, the second wire is folded back at the first end (the upper end) and then woven from the first end (the upper end) toward the second end (the lower end) in the axial direction. The stent 2 according to the fourth embodiment is formed by combining the first wire in FIGS. 18A and 18B (or FIG. 19) and the second wire in FIGS. 20A and 20B (or FIG. 21). However, the first wire and the second wire are independent from each other and no hooking portion 22 is formed between the first wire and the second wire in the fourth embodiment. In other words, only the crossing portions 21 are formed between the first wire and the second wire.

FIG. 21 schematically illustrates a result of weaving the second wire forming the stent 2 according to the fourth embodiment back and forth along the axial direction. This drawing is obtained by superimposing FIG. 20A representing the forward path and FIG. 20B representing the backward path on each other. In the stent 2 (the second wire), the stretch assisting region 23 including a larger number of the crossing portions 21 than the hooking portions 22 and the contraction assisting region 24 including a larger number of the hooking portions 22 than the crossing portions 21 are separately disposed along the circumferential direction. In the fourth embodiment of FIG. 21, the circumference of the stent 2 having a tubular shape is divided into the one stretch assisting region 23 and the one contraction assisting region 24.

In the stretch assisting region 23, at least two stretch assisting columns each of which includes a larger number of the crossing portions 21 than the hooking portions 22 in the axial direction (vertical direction) of the stent 2 are continuously disposed in the circumferential direction (horizontal direction) of the stent 2. In the example illustrated, two columns of the 0th (12th) column and the 1st column continuous in the circumferential direction are the stretch assisting columns. In the contraction assisting region 24, at least one or preferably a plurality of contraction assisting columns each of which includes a larger number of the hooking portions 22 than the crossing portions 21 in the axial direction (vertical direction) of the stent 2 are disposed. In the example illustrated, ten columns of the 2nd column to the 11th column continuous in the circumferential direction are the contraction assisting columns. In this manner, the contraction assisting region 24 including the plurality of contraction assisting columns is formed in a region in the circumferential direction excluding the stretch assisting region 23 including the at least two continuously-disposed stretch assisting columns. In a manner similar to FIG. 4, when the stent 2 is equally divided in the circumferential direction by a plane including the axis of the stent 2 and a first equally-divided region includes the at least two continuously-disposed stretch assisting columns (that is, the stretch assisting region 23), a second equally-divided region (including the contraction assisting region 24 only) includes a majority of the hooking portions 22.

In the fourth embodiment of FIG. 21, all the hooking portions 22 are provided in the contraction assisting region 24. However, in order to produce a significant difference in stretchability between the stretch assisting region 23 and the contraction assisting region 24, it is enough to provide a majority of the hooking portions 22 in the contraction assisting region 24 (or provide less than half of the hooking portions 22 in the stretch assisting region 23). For example, 70% or more of the hooking portions 22 may be provided in the contraction assisting region 24, or 90% or more of the hooking portions 22 may be provided in the contraction assisting region 24.

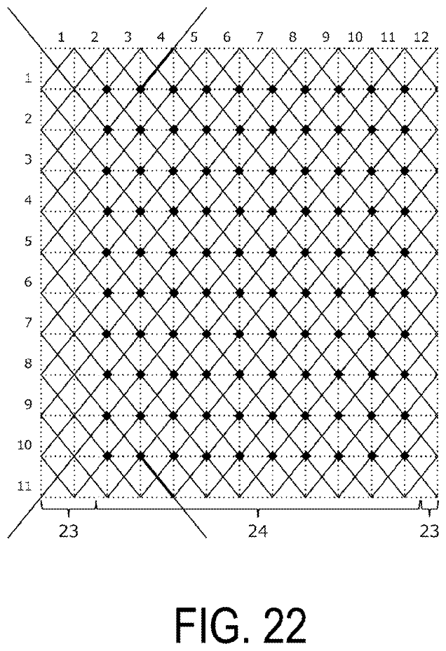

FIGS. 20A and 20B schematically illustrate the stent 2 according to the fourth embodiment formed by combining the first wire and the second wire. This drawing is obtained by superimposing FIG. 19 representing the first wire and FIG. 21 representing the second wire on each other. In the stent 2, the stretch assisting region 23, which is an overlapping region between the first stretch assisting region 23 of the first wire (FIG. 19) and the second stretch assisting region 23 of the second wire (FIG. 21), and the contraction assisting region 24, which is an overlapping region between the first contraction assisting region 24 of the first wire (FIG. 19) and the second contraction assisting region 24 of the second wire (FIG. 21), are separately disposed along the circumferential direction. In the fourth embodiment of FIG. 22, the circumference of the stent 2 having a tubular shape is divided into the one stretch assisting region 23 and the one contraction assisting region 24.

In the stretch assisting region 23, at least three stretch assisting columns each of which includes a larger number of the crossing portions 21 than the hooking portions 22 in the axial direction (vertical direction) of the stent 2 are continuously disposed in the circumferential direction (horizontal direction) of the stent 2. As a result of combining the two wires, in addition to the 12 columns of the 0th (12th) column to the 11th column constituting the grid points, additional 12 columns including the crossing portions 21 between the first wire and the second wire are formed, each of which is located between corresponding adjacent two of the 0th (12th) to the 11th columns. That is, in the example of FIG. 22, a total of 24 columns are formed. In the example illustrated, five columns of the 11.5th column, the 0th (12th) column, the 0.5th column, the 1st column, and the 1.5th column continuous in the circumferential direction are the stretch assisting columns. In the contraction assisting region 24, at least one or preferably a plurality of contraction assisting columns each of which includes a larger number of the hooking portions 22 than the crossing portions 21 in the axial direction (vertical direction) of the stent 2 are disposed. In the example illustrated, ten columns of the 2nd column to the 11th column are the contraction assisting columns. The 2.5th column, the 3.5th column, the 4.5th column, the 5.5th column, the 6.5th column, the 7.5th column, the 8.5th column, the 9.5th column, and the 10.5th column formed between the respective contraction assisting columns by the crossing portions 21 between the first wire and the second wire are separate stretch assisting columns that are not continuous in the circumferential direction and thus do not constitute the stretch assisting region 23. In this manner, the contraction assisting region 24 including the plurality of contraction assisting columns is formed in a region in the circumferential direction excluding the stretch assisting region 23 including the at least three continuously-disposed stretch assisting columns. In a manner similar to FIG. 4, when the stent 2 is equally divided in the circumferential direction by a plane including the axis of the stent 2 and a first equally-divided region includes the at least three continuously-disposed stretch assisting columns (that is, the stretch assisting region 23), a second equally-divided region (including the contraction assisting region 24 only) includes a majority of the hooking portions 22.

The stretch assisting region 23 and the contraction assisting region 24 extend along the axial direction (vertical direction in FIG. 22) of the stent 2 having a tubular shape. The stretch assisting region 23 including a large number of the crossing portions 21 is easy to stretch and hard to contract along the axial direction of the stent 2 having a tubular shape. The contraction assisting region 24 including a large number of the hooking portions 22 is easy to contract and hard to stretch along the axial direction of the stent 2 having a tubular shape. For this reason, when the stent graft 1 is placed at a dilation target site having a curvature, the contraction assisting region 24 which is easy to contract is disposed on the inner side having a large curvature and the stretch assisting region 23 which is easy to stretch is disposed on the outer side having a small curvature, so that the stent graft 1 can be easily curved.

In the fourth embodiment of FIG. 22, all the hooking portions 22 are provided in the contraction assisting region 24. However, in order to produce a significant difference in stretchability between the stretch assisting region 23 and the contraction assisting region 24, it is enough to provide a majority of the hooking portions 22 in the contraction assisting region 24 (or provide less than half of the hooking portions 22 in the stretch assisting region 23). For example, 70% or more of the hooking portions 22 may be provided in the contraction assisting region 24, or 90% or more of the hooking portions 22 may be provided in the contraction assisting region 24.

In the fourth embodiment of FIG. 22, the first stretch assisting region 23 of the first wire and the second stretch assisting region 23 of the second wire completely coincide with each other, and the first contraction assisting region 24 of the first wire and the second contraction assisting region 24 of the second wire completely coincide with each other. However, it is enough that the first stretch assisting region 23 and the second stretch assisting region 23 overlap with each other and the first contraction assisting region 24 and the second contraction assisting region 24 overlap with each other.

As described above, in the fourth embodiment, no hooking portion 22 is formed between the first wire and the second wire, and only the crossing portions 21 are formed between the first wire and the second wire. Thus, the first wire and the second wire also form the crossing portions 21 in the overlapping region between the first contraction assisting region 24 and the second contraction assisting region 24. As a result, in the contraction assisting region 24 obtained by combining the first wire and the second wire, substantially equal numbers of the crossing portions 21 and the hooking portions 22 are formed. However, as illustrated in FIG. 19 (the first wire) and FIG. 21 (the second wire), when a focus is placed on each wire, a larger number of the hooking portions 22 than the crossing portions 21 are formed in the contraction assisting region 24.

As described in the first embodiment, the number of the hooking portions 22 is (X−Z)(Y−1), and the number of the crossing portions 21 is XY+(Y−1)Z. In the fourth embodiment of FIG. 22, X=12, Y=11, and Z=2 (each of the first wire and the second wire is woven back and forth once), and thus the number of the hooking portions 22 is (12 −2)(11 −1)=100, and the number of the crossing portions 21 is 12×11+(11 −1)×2=152. Additionally, in the fourth embodiment described above, the number X of the positions (grid points) in the circumferential direction at which each wire can form the crossing portion 21 or the hooking portion 22 is an even number (12), and the one stretch assisting region 23 and the one contraction assisting region 24 are disposed along the circumferential direction.

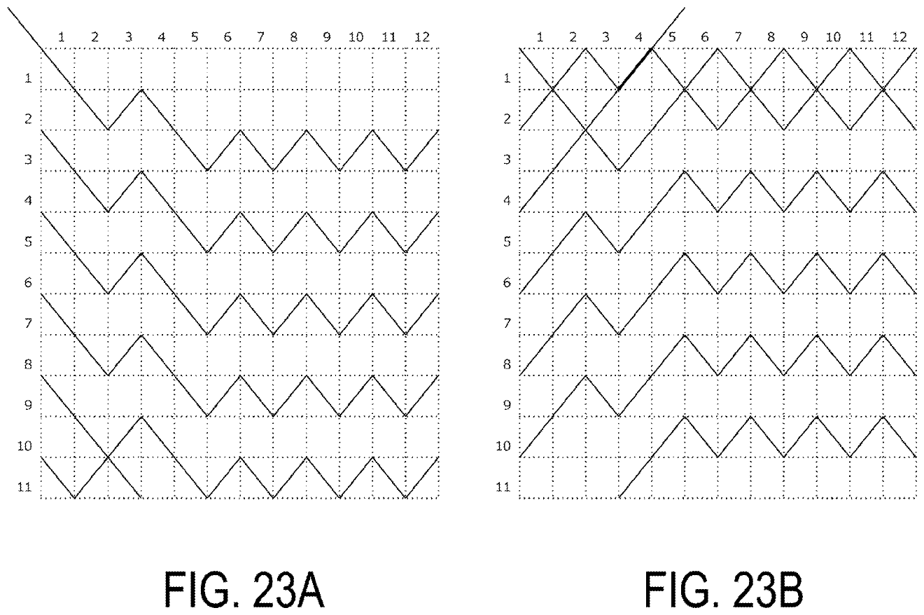

FIGS. 23A and 23B schematically illustrate a weaving method of the first wire forming the stent 2 according to a fifth embodiment. In the present embodiment, the total number X of the grid points in the circumferential direction is 12, and the total number Y of the grid points in the axial direction is 11. FIG. 23A illustrates a state in which the first wire forming the stent 2 is woven from a first end (the upper end) toward a second end (the lower end) in the axial direction. FIG. 23B illustrates a state in which, following FIG. 23A, the first wire is folded back at the second end (the lower end) and then woven from the second end (the lower end) toward the first end (the upper end) in the axial direction.

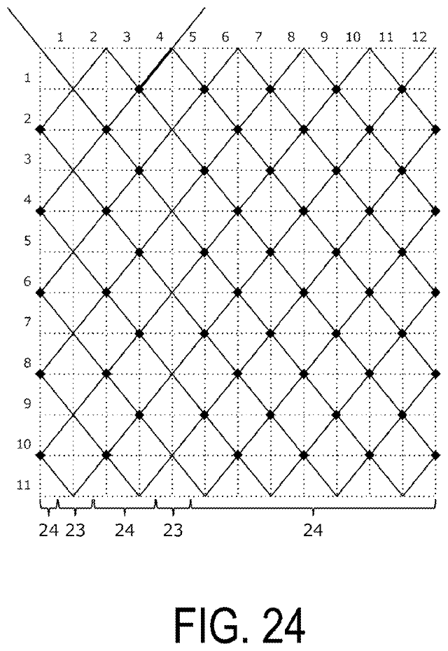

FIG. 24 schematically illustrates a result of weaving the first wire forming the stent 2 according to the fifth embodiment back and forth along the axial direction. This drawing is obtained by superimposing FIG. 23A representing the forward path and FIG. 23B representing the backward path on each other. In the stent 2 (the first wire), the stretch assisting regions 23 each including a larger number of the crossing portions 21 than the hooking portions 22 and the contraction assisting regions 24 each including a larger number of the hooking portions 22 than the crossing portions 21 are separately disposed along the circumferential direction. In the fifth embodiment of FIG. 24, the circumference of the stent 2 having a tubular shape is divided into the two stretch assisting regions 23 and the two contraction assisting regions 24.

The stretch assisting regions 23 and the contraction assisting regions 24 extend along the axial direction (vertical direction in FIG. 24) of the stent 2 having a tubular shape. Each stretch assisting region 23 in which the crossing portion 21 is superior in numbers is easy to stretch and hard to contract along the axial direction of the stent 2 having a tubular shape. Each contraction assisting region 24 in which the hooking portion 22 is superior in numbers is easy to contract and hard to stretch along the axial direction of the stent 2 having a tubular shape. For this reason, when the stent graft 1 is placed at a dilation target site having a curvature, the contraction assisting regions 24 which are easy to contract are disposed on the inner side having a large curvature and the stretch assisting regions 23 which are easy to stretch are disposed on the outer side having a small curvature, so that the stent graft 1 can be easily curved. In particular, the stretch assisting region 23 (and the contraction assisting region 24) formed as one region in the first embodiment, the second embodiment, and the fourth embodiment is divided into a plurality of regions and distributed along the circumferential direction in the present embodiment. Thus, even when the stent graft 1 turns around the axial direction while being placed at a dilation target site having a curvature, the stent graft 1 can be stably curved.

In the fifth embodiment of FIG. 24, all the hooking portions 22 are provided in the contraction assisting regions 24. However, in order to produce a significant difference in stretchability between the stretch assisting regions 23 and the contraction assisting regions 24, it is enough to provide a majority of the hooking portions 22 in the contraction assisting regions 24 (or provide less than half of the hooking portions 22 in the stretch assisting regions 23). For example, 70% or more of the hooking portions 22 may be provided in the contraction assisting regions 24, or 90% or more of the hooking portions 22 may be provided in the contraction assisting regions 24.

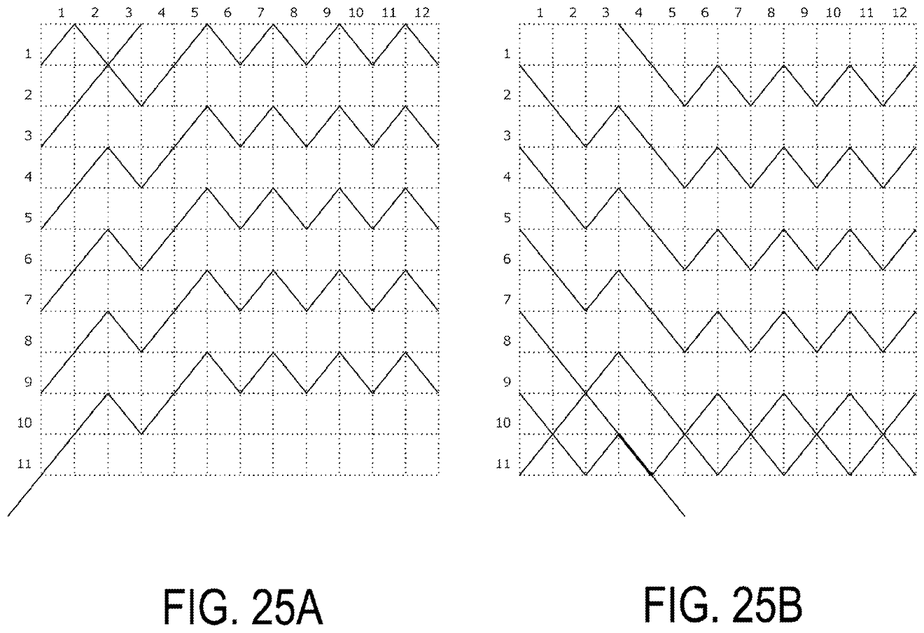

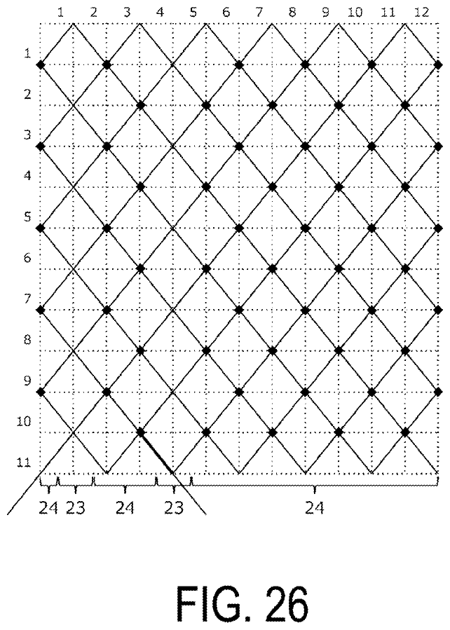

FIG. 25 schematically illustrate a weaving method of the second wire forming the stent 2 according to the fifth embodiment. FIG. 25A illustrates a state in which the second wire forming the stent 2 is woven from the second end (the lower end) toward the first end (the upper end) in the axial direction. FIG. 25B illustrates a state in which, following FIG. 25A, the second wire is folded back at the first end (the upper end) and then woven from the first end (the upper end) toward the second end (the lower end) in the axial direction. The stent 2 according to the fifth embodiment is formed by combining the first wire in FIGS. 23A and 23B (or FIG. 24) and the second wire in FIGS. 25A and 215B (or FIG. 26). However, the first wire and the second wire are independent from each other and no hooking portion 22 is formed between the first wire and the second wire in the fifth embodiment. In other words, only the crossing portions 21 are formed between the first wire and the second wire.

FIG. 26 schematically illustrates a result of weaving the second wire forming the stent 2 according to the fifth embodiment back and forth along the axial direction. This drawing is obtained by superimposing FIG. 25A representing the forward path and FIG. 25B representing the backward path on each other. In the stent 2 (the second wire), the stretch assisting regions 23 each including a larger number of the crossing portions 21 than the hooking portions 22 and the contraction assisting regions 24 each including a larger number of the hooking portions 22 than the crossing portions 21 are separately disposed along the circumferential direction. In the fifth embodiment of FIG. 26, the circumference of the stent 2 having a tubular shape is divided into the two stretch assisting regions 23 and the two contraction assisting regions 24.

In the fifth embodiment of FIG. 26, all the hooking portions 22 are provided in the contraction assisting regions 24. However, in order to produce a significant difference in stretchability between the stretch assisting regions 23 and the contraction assisting regions 24, it is enough to provide a majority of the hooking portions 22 in the contraction assisting regions 24 (or provide less than half of the hooking portions 22 in the stretch assisting regions 23). For example, 70% or more of the hooking portions 22 may be provided in the contraction assisting regions 24, or 90% or more of the hooking portions 22 may be provided in the contraction assisting regions 24.

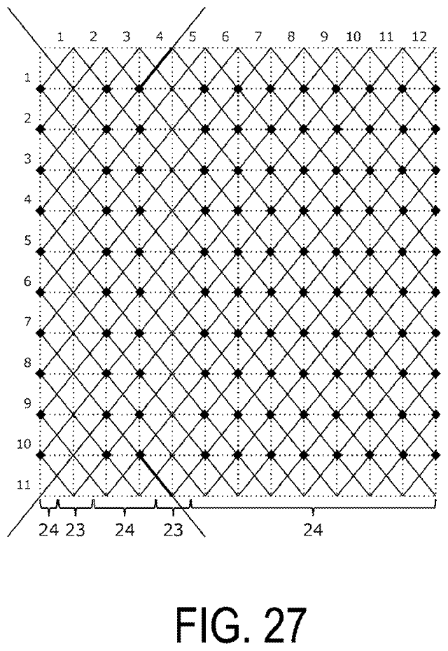

FIG. 27 schematically illustrates the stent 2 according to the fifth embodiment formed by combining the first wire and the second wire. This drawing is obtained by superimposing FIG. 24 representing the first wire and FIG. 26 representing the second wire on each other. In the stent 2, the stretch assisting regions 23, which are overlapping regions between the first stretch assisting regions 23 of the first wire (FIG. 24) and the second stretch assisting regions 23 of the second wire (FIG. 26), and the contraction assisting regions 24, which are overlapping regions between the first contraction assisting regions 24 of the first wire (FIG. 24) and the second contraction assisting regions 24 of the second wire (FIG. 26), are separately disposed along the circumferential direction. In the fifth embodiment of FIG. 27, the circumference of the stent 2 having a tubular shape is divided into the two stretch assisting regions 23 and the two contraction assisting regions 24.

In each stretch assisting region 23, at least three stretch assisting columns each of which includes a larger number of the crossing portions 21 than the hooking portions 22 in the axial direction (vertical direction) of the stent 2 are continuously disposed in the circumferential direction (horizontal direction) of the stent 2. As a result of combining the two wires, in addition to the 12 columns of the 0th (12th) column to the 11th column constituting the grid points, additional 12 columns including the crossing portions 21 between the first wire and the second wire are formed, each of which is located between corresponding adjacent two of the 0th (12th) to the 11th columns. That is, in the example of FIG. 27, a total of 24 columns are formed. In the example illustrated, three columns of the 0.5th column, the 1st column, and the 1.5th column continuous in the circumferential direction, and three columns of the 3.5th column, the 4th column, and the 4.5th column continuous in the circumferential direction are the stretch assisting columns. In this manner, the plurality of stretch assisting regions 23, each of which includes the at least three continuously-disposed stretch assisting columns, are disposed separately from each other in the circumferential direction. In each contraction assisting region 24, at least one or preferably a plurality of contraction assisting columns each of which includes a larger number of the hooking portions 22 than the crossing portions 21 in the axial direction (vertical direction) of the stent 2 are disposed. In the example illustrated, ten columns of the 2nd column, the 3rd column, and the 5th column to the 12th column are the contraction assisting columns. The 2.5th column, the 5.5th column, the 6.5th column, the 7.5th column, the 8.5th column, the 9.5th column, the 10.5th column, and the 11.5th column formed between adjacent contraction assisting columns by the crossing portions 21 between the first wire and the second wire are separate stretch assisting columns that are not continuous in the circumferential direction and thus do not constitute the stretch assisting regions 23. In this manner, the contraction assisting regions 24 each including the plurality of contraction assisting columns are formed in regions in the circumferential direction excluding the stretch assisting regions 23 each including the at least three continuously-disposed stretch assisting columns. In a manner similar to FIG. 4, when the stent 2 is equally divided in the circumferential direction by a plane including the axis of the stent 2 and a first equally-divided region includes the at least three continuously-disposed stretch assisting columns (that is, the stretch assisting region 23), a second equally-divided region (including the contraction assisting region 24 only) includes a majority of the hooking portions 22.

The stretch assisting regions 23 and the contraction assisting regions 24 extend along the axial direction (vertical direction in FIG. 27) of the stent 2 having a tubular shape. Each stretch assisting region 23 including a large number of the crossing portions 21 is easy to stretch and hard to contract along the axial direction of the stent 2 having a tubular shape. Each contraction assisting region 24 including a large number of the hooking portions 22 is easy to contract and hard to stretch along the axial direction of the stent 2 having a tubular shape. For this reason, when the stent graft 1 is placed at a dilation target site having a curvature, the contraction assisting regions 24 which are easy to contract are disposed on the inner side having a large curvature and the stretch assisting regions 23 which are easy to stretch are disposed on the outer side having a small curvature, so that the stent graft 1 can be easily curved. In particular, the stretch assisting region 23 (and the contraction assisting region 24) formed as one region in the first embodiment, the second embodiment, and the fourth embodiment is divided into a plurality of regions and distributed along the circumferential direction in the present embodiment. Thus, even when the stent graft 1 turns around the axial direction while being placed at a dilation target site having a curvature, the stent graft 1 can be stably curved.

In the fifth embodiment of FIG. 27, all the hooking portions 22 are provided in the contraction assisting regions 24. However, in order to produce a significant difference in stretchability between the stretch assisting regions 23 and the contraction assisting regions 24, it is enough to provide a majority of the hooking portions 22 in the contraction assisting regions 24 (or provide less than half of the hooking portions 22 in the stretch assisting regions 23). For example, 70% or more of the hooking portions 22 may be provided in the contraction assisting regions 24, or 90% or more of the hooking portions 22 may be provided in the contraction assisting regions 24.

In the fifth embodiment of FIG. 27, the first stretch assisting regions 23 of the first wire and the second stretch assisting regions 23 of the second wire completely coincide with each other, and the first contraction assisting regions 24 of the first wire and the second contraction assisting regions 24 of the second wire completely coincide with each other. However, it is enough that the first stretch assisting regions 23 and the second stretch assisting regions 23 overlap with each other and the first contraction assisting regions 24 and the second contraction assisting regions 24 overlap with each other.

As described above, in the fifth embodiment, no hooking portion 22 is formed between the first wire and the second wire, and only the crossing portions 21 are formed between the first wire and the second wire. Thus, the first wire and the second wire also form the crossing portions 21 in the overlapping regions between the first contraction assisting regions 24 and the second contraction assisting regions 24. As a result, in the contraction assisting regions 24 obtained by combining the first wire and the second wire, substantially equal numbers of the crossing portions 21 and the hooking portions 22 are formed. However, as illustrated in FIG. 24 (the first wire) and FIG. 26 (the second wire), when a focus is placed on each wire, a larger number of the hooking portions 22 than the crossing portions 21 are formed in the contraction assisting regions 24.

As described in the first embodiment, the number of the hooking portions 22 is (X−Z)(Y−1), and the number of the crossing portions 21 is XY+(Y−1)Z. In the fifth embodiment of FIG. 27, X=12, Y=11, and Z=2 (each of the first wire and the second wire is woven back and forth once), and thus the number of the hooking portions 22 is (12 −2)(11 −1)=100, and the number of the crossing portions 21 is 12×11+(11 −1)×2=152. Additionally, in the fifth embodiment described above, the number X of the positions (grid points) in the circumferential direction at which each wire can form the crossing portion 21 or the hooking portion 22 is an even number (12), and the plurality of stretch assisting regions 23 are disposed separately from each other along the circumferential direction, and the plurality of contraction assisting regions 24 are disposed separately from each other along the circumferential direction.

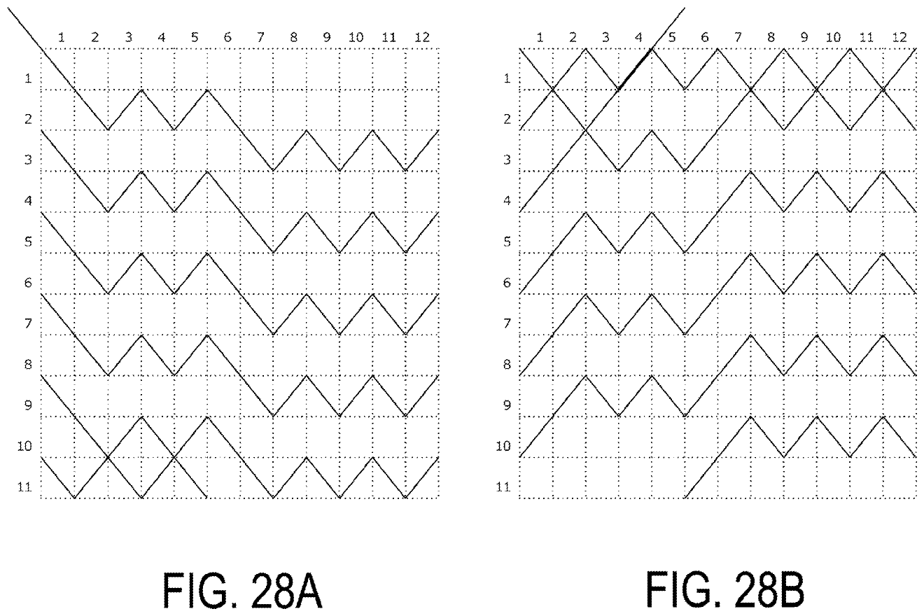

FIGS. 28A and 28B schematically illustrate a weaving method of the first wire forming the stent 2 according to a sixth embodiment. In the present embodiment, the total number X of the grid points in the circumferential direction is 12, and the total number Y of the grid points in the axial direction is 11. FIG. 28A illustrates a state in which the first wire forming the stent 2 is woven from a first end (the upper end) toward a second end (the lower end) in the axial direction. FIG. 28B illustrates a state in which, following FIG. 28A, the first wire is folded back at the second end (the lower end) and then woven from the second end (the lower end) toward the first end (the upper end) in the axial direction.

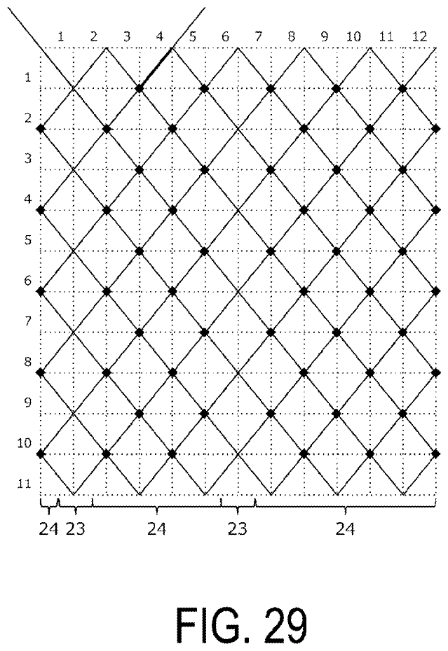

FIG. 29 schematically illustrates a result of weaving the first wire forming the stent 2 according to the sixth embodiment back and forth along the axial direction. This drawing is obtained by superimposing FIG. 28A representing the forward path and FIG. 28B representing the backward path on each other. In the stent 2 (the first wire), the stretch assisting regions 23 each including a larger number of the crossing portions 21 than the hooking portions 22 and the contraction assisting regions 24 each including a larger number of the hooking portions 22 than the crossing portions 21 are separately disposed along the circumferential direction. In the sixth embodiment of FIG. 29, the circumference of the stent 2 having a tubular shape is divided into the two stretch assisting regions 23 and the two contraction assisting regions 24.

The stretch assisting regions 23 and the contraction assisting regions 24 extend along the axial direction (vertical direction in FIG. 29) of the stent 2 having a tubular shape. Each stretch assisting region 23 in which the crossing portion 21 is superior in numbers is easy to stretch and hard to contract along the axial direction of the stent 2 having a tubular shape. Each contraction assisting region 24 in which the hooking portion 22 is superior in numbers is easy to contract and hard to stretch along the axial direction of the stent 2 having a tubular shape. For this reason, when the stent graft 1 is placed at a dilation target site having a curvature, the contraction assisting regions 24 which are easy to contract are disposed on the inner side having a large curvature and the stretch assisting regions 23 which are easy to stretch are disposed on the outer side having a small curvature, so that the stent graft 1 can be easily curved. In particular, the stretch assisting region 23 (and the contraction assisting region 24) formed as one region in the first embodiment, the second embodiment, and the fourth embodiment is divided into a plurality of regions and distributed along the circumferential direction in the present embodiment. Thus, even when the stent graft 1 turns around the axial direction while being placed at a dilation target site having a curvature, the stent graft 1 can be stably curved.

Further, since the spacing between the stretch assisting regions 23 in the circumferential direction is larger than that in the fifth embodiment (FIG. 24), a permissible turning amount of the stent graft 1 can be increased. In this manner, when the number X of the positions (grid points) in the circumferential direction at which each wire can form the crossing portion 21 or the hooking portion 22 is an even number equal to or greater than 12, the spacing between the plurality of stretch assisting regions 23 in the circumferential direction can be changed.

In the sixth embodiment of FIG. 29, all the hooking portions 22 are provided in the contraction assisting regions 24. However, in order to produce a significant difference in stretchability between the stretch assisting regions 23 and the contraction assisting regions 24, it is enough to provide a majority of the hooking portions 22 in the contraction assisting regions 24 (or provide less than half of the hooking portions 22 in the stretch assisting regions 23). For example, 70% or more of the hooking portions 22 may be provided in the contraction assisting regions 24, or 90% or more of the hooking portions 22 may be provided in the contraction assisting regions 24.

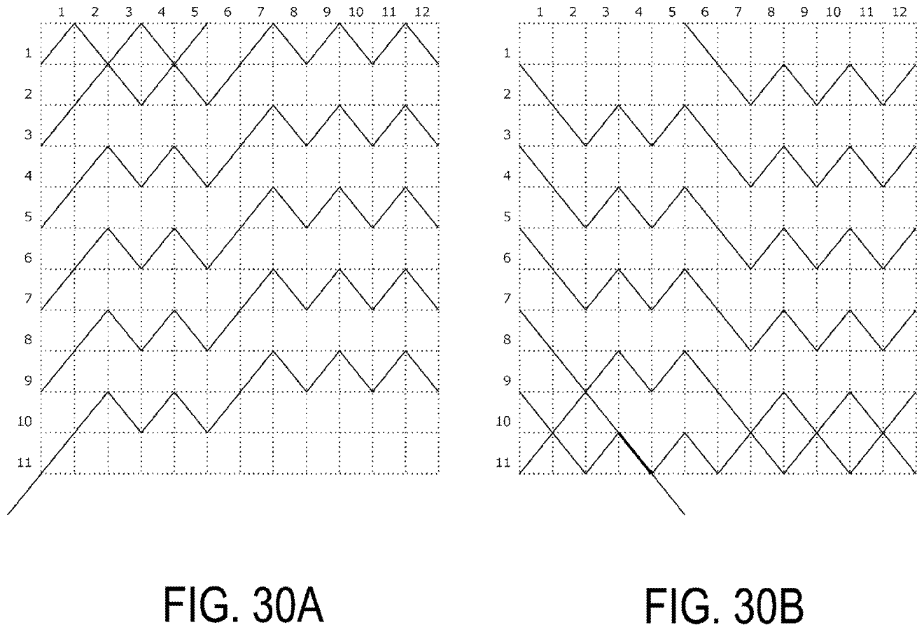

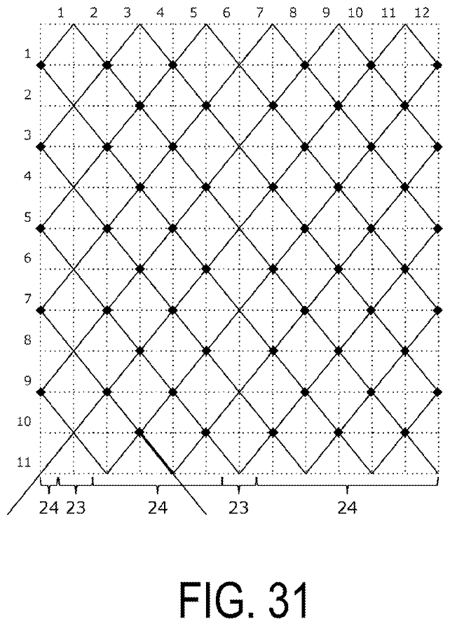

FIGS. 30A and 30B schematically illustrate a weaving method of the second wire forming the stent 2 according to the sixth embodiment. FIG. 30A illustrates a state in which the second wire forming the stent 2 is woven from the second end (the lower end) toward the first end (the upper end) in the axial direction. FIG. 30B illustrates a state in which, following FIG. 30A, the second wire is folded back at the first end (the upper end) and then woven from the first end (the upper end) toward the second end (the lower end) in the axial direction. The stent 2 according to the sixth embodiment is formed by combining the first wire in FIGS. 28A and 28B (or FIG. 29) and the second wire in FIGS. 30A and 30B (or FIG. 31). However, the first wire and the second wire are independent from each other and no hooking portion 22 is formed between the first wire and the second wire in the sixth embodiment. In other words, only the crossing portions 21 are formed between the first wire and the second wire.

FIG. 31 schematically illustrates a result of weaving the second wire forming the stent 2 according to the sixth embodiment back and forth along the axial direction. This drawing is obtained by superimposing FIG. 30A representing the forward path and FIG. 30B representing the backward path on each other. In the stent 2 (the second wire), the stretch assisting regions 23 each including a larger number of the crossing portions 21 than the hooking portions 22 and the contraction assisting regions 24 each including a larger number of the hooking portions 22 than the crossing portions 21 are separately disposed along the circumferential direction. In the sixth embodiment of FIG. 31, the circumference of the stent 2 having a tubular shape is divided into the two stretch assisting regions 23 and the two contraction assisting regions 24.

In the sixth embodiment of FIG. 31, all the hooking portions 22 are provided in the contraction assisting regions 24. However, in order to produce a significant difference in stretchability between the stretch assisting regions 23 and the contraction assisting regions 24, it is enough to provide a majority of the hooking portions 22 in the contraction assisting regions 24 (or provide less than half of the hooking portions 22 in the stretch assisting regions 23). For example, 70% or more of the hooking portions 22 may be provided in the contraction assisting regions 24, or 90% or more of the hooking portions 22 may be provided in the contraction assisting regions 24.

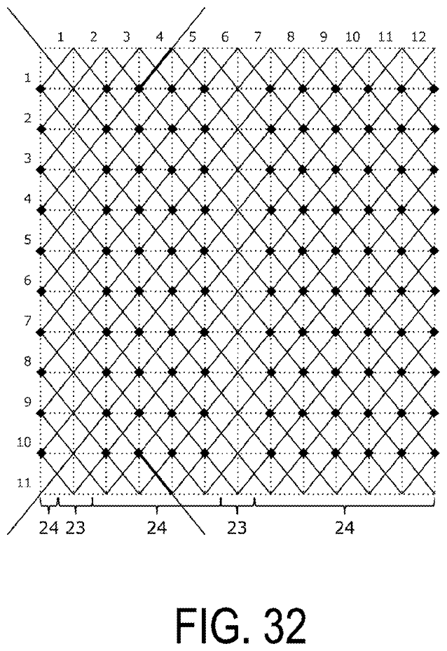

FIG. 32 schematically illustrates the stent 2 according to the sixth embodiment formed by combining the first wire and the second wire. This drawing is obtained by superimposing FIG. 29 representing the first wire and FIG. 31 representing the second wire on each other. In the stent 2, the stretch assisting regions 23, which are overlapping regions between the first stretch assisting regions 23 of the first wire (FIG. 29) and the second stretch assisting regions 23 of the second wire (FIG. 31), and the contraction assisting regions 24, which are overlapping regions between the first contraction assisting regions 24 of the first wire (FIG. 29) and the second contraction assisting regions 24 of the second wire (FIG. 31), are separately disposed along the circumferential direction. In the sixth embodiment of FIG. 32, the circumference of the stent 2 having a tubular shape is divided into the two stretch assisting regions 23 and the two contraction assisting regions 24.

In each stretch assisting region 23, at least three stretch assisting columns each of which includes a larger number of the crossing portions 21 than the hooking portions 22 in the axial direction (vertical direction) of the stent 2 are continuously disposed in the circumferential direction (horizontal direction) of the stent 2. As a result of combining the two wires, in addition to the 12 columns of the 0th (12th) column to the 11th column constituting the grid points, additional 12 columns including the crossing portions 21 between the first wire and the second wire are formed, each of which is located between corresponding adjacent two of the 0th (12th) to the 11th columns. That is, in the example of FIG. 32, a total of 24 columns are formed. In the example illustrated, three columns of the 0.5th column, the 1st column, and the 1.5th column continuous in the circumferential direction, and three columns of the 5.5th column, the 6th column, and the 6.5th column continuous in the circumferential direction are the stretch assisting columns. In this manner, the plurality of stretch assisting regions 23, each of which includes the at least three continuously-disposed stretch assisting columns, are disposed separately from each other in the circumferential direction. In each contraction assisting region 24, at least one or preferably a plurality of contraction assisting columns each of which includes a larger number of the hooking portions 22 than the crossing portions 21 in the axial direction (vertical direction) of the stent 2 are disposed. In the example illustrated, ten columns of the 2nd column to the 5th column, and the 7th column to the 12th column are the contraction assisting columns. The 2.5th column, the 3.5th column, the 4.5th column, the 7.5th column, the 8.5th column, the 9.5th column, the 10.5th column, and the 11.5th column formed between adjacent contraction assisting columns by the crossing portions 21 between the first wire and the second wire are separate stretch assisting columns that are not continuous in the circumferential direction and thus do not constitute the stretch assisting regions 23. In this manner, the contraction assisting regions 24 each including the plurality of contraction assisting columns are formed in regions in the circumferential direction excluding the stretch assisting regions 23 each including the at least three continuously-disposed stretch assisting columns. In a manner similar to FIG. 4, when the stent 2 is equally divided in the circumferential direction by a plane including the axis of the stent 2 and a first equally-divided region includes the at least three continuously-disposed stretch assisting columns (that is, the stretch assisting region 23), a second equally-divided region (including the contraction assisting region 24 only) includes a majority of the hooking portions 22.

The stretch assisting regions 23 and the contraction assisting regions 24 extend along the axial direction (vertical direction in FIG. 32) of the stent 2 having a tubular shape. Each stretch assisting region 23 including a large number of the crossing portions 21 is easy to stretch and hard to contract along the axial direction of the stent 2 having a tubular shape. Each contraction assisting region 24 including a large number of the hooking portions 22 is easy to contract and hard to stretch along the axial direction of the stent 2 having a tubular shape. For this reason, when the stent graft 1 is placed at a dilation target site having a curvature, the contraction assisting regions 24 which are easy to contract are disposed on the inner side having a large curvature and the stretch assisting regions 23 which are easy to stretch are disposed on the outer side having a small curvature, so that the stent graft 1 can be easily curved. In particular, the stretch assisting region 23 (and the contraction assisting region 24) formed as one region in the first embodiment, the second embodiment, and the fourth embodiment is divided into a plurality of regions and distributed along the circumferential direction in the present embodiment. Thus, even when the stent graft 1 turns around the axial direction while being placed at a dilation target site having a curvature, the stent graft 1 can be stably curved.

Further, since the spacing between the stretch assisting regions 23 in the circumferential direction is larger than that in the fifth embodiment (FIG. 27), a permissible turning amount of the stent graft 1 can be increased. In this manner, when the number X of the positions (grid points) in the circumferential direction at which each wire can form the crossing portion 21 or the hooking portion 22 is an even number equal to or greater than 12, the spacing between the plurality of stretch assisting regions 23 in the circumferential direction can be changed.

In the sixth embodiment of FIG. 32, all the hooking portions 22 are provided in the contraction assisting regions 24. However, in order to produce a significant difference in stretchability between the stretch assisting regions 23 and the contraction assisting regions 24, it is enough to provide a majority of the hooking portions 22 in the contraction assisting regions 24 (or provide less than half of the hooking portions 22 in the stretch assisting regions 23). For example, 70% or more of the hooking portions 22 may be provided in the contraction assisting regions 24, or 90% or more of the hooking portions 22 may be provided in the contraction assisting regions 24.

In the sixth embodiment of FIG. 32, the first stretch assisting regions 23 of the first wire and the second stretch assisting regions 23 of the second wire completely coincide with each other, and the first contraction assisting regions 24 of the first wire and the second contraction assisting regions 24 of the second wire completely coincide with each other. However, it is enough that the first stretch assisting regions 23 and the second stretch assisting regions 23 overlap with each other and the first contraction assisting regions 24 and the second contraction assisting regions 24 overlap with each other.

As described above, in the sixth embodiment, no hooking portion 22 is formed between the first wire and the second wire, and only the crossing portions 21 are formed between the first wire and the second wire. Thus, the first wire and the second wire also form the crossing portions 21 in the overlapping regions between the first contraction assisting regions 24 and the second contraction assisting regions 24. As a result, in the contraction assisting regions 24 obtained by combining the first wire and the second wire, substantially equal numbers of the crossing portions 21 and the hooking portions 22 are formed. However, as illustrated in FIG. 29 (the first wire) and FIG. 31 (the second wire), when a focus is placed on each wire, a larger number of the hooking portions 22 than the crossing portions 21 are formed in the contraction assisting regions 24.

As described in the first embodiment, the number of the hooking portions 22 is (X−Z)(Y−1), and the number of the crossing portions 21 is XY+(Y−1)Z. In the sixth embodiment of FIG. 32, X=12, Y=11, and Z=2 (each of the first wire and the second wire is woven back and forth once), and thus the number of the hooking portions 22 is (12 −2)(11 −1)=100, and the number of the crossing portions 21 is 12×11+(11 −1)×2=152. Additionally, in the sixth embodiment described above, the number X of the positions (grid points) in the circumferential direction at which each wire can form the crossing portion 21 or the hooking portion 22 is an even number (12), and the plurality of stretch assisting regions 23 are disposed separately from each other along the circumferential direction, and the plurality of contraction assisting regions 24 are disposed separately from each other along the circumferential direction.

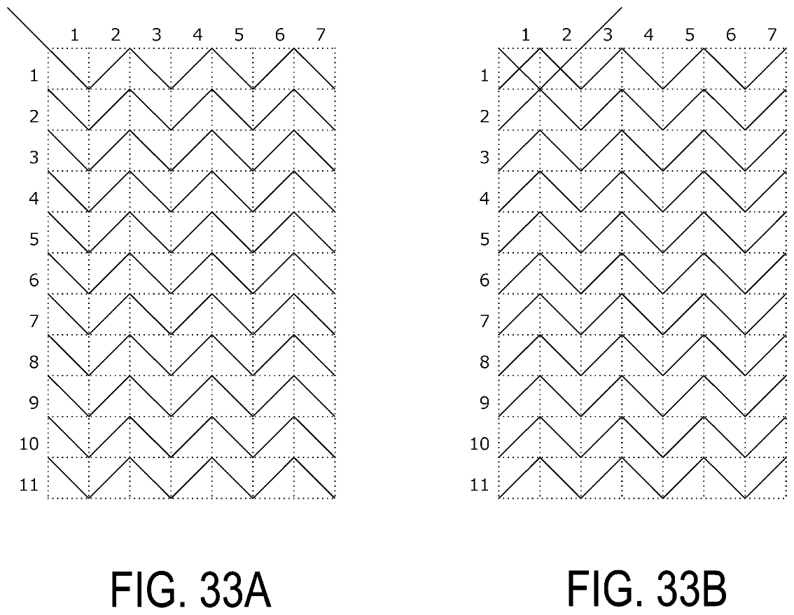

FIGS. 33A and 33B schematically illustrate a weaving method of a single wire forming the stent 2 according to a seventh embodiment. In the present embodiment, the total number X of the grid points in the circumferential direction is 7, and the total number Y of the grid points in the axial direction is 11. FIG. 33A illustrates a state in which a single wire forming the stent 2 is woven from a first end (the upper end) toward a second end (the lower end) in the axial direction. FIG. 33B illustrates a state in which, following FIG. 33A, the single wire is folded back at the second end (the lower end) and then woven from the second end (the lower end) toward the first end (the upper end) in the axial direction.

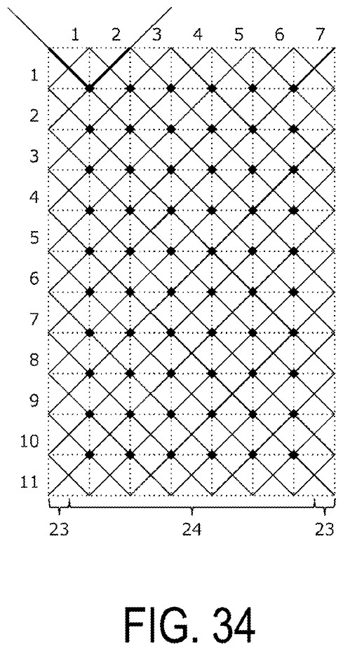

FIG. 34 schematically illustrates a result of weaving the single wire forming the stent 2 according to the seventh embodiment back and forth along the axial direction. This drawing is obtained by superimposing FIG. 33A representing the forward path and FIG. 33B representing the backward path on each other. In the stent 2, the contraction assisting region 24 in which a majority of the hooking portions 22 are provided, and the stretch assisting region 23 in which the crossing portions 21 are provided together with less than half (zero in the example of FIG. 34) of the hooking portions 22 are separately disposed in the circumferential direction. In the seventh embodiment of FIG. 34, the circumference of the stent 2 having a tubular shape is divided into the one stretch assisting region 23 and the one contraction assisting region 24.

In the stretch assisting region 23, at least three stretch assisting columns each of which includes a larger number of the crossing portions 21 than the hooking portions 22 in the axial direction (vertical direction) of the stent 2 are continuously disposed in the circumferential direction (horizontal direction) of the stent 2. In FIG. 34, in addition to the seven columns of the 0th (7th) column to the 6th column constituting the grid points, additional seven columns including the crossing portions 21 are formed, each of which is located between corresponding adjacent two of the 0th (7th) to the 6th columns. That is, in the example of FIG. 34, a total of 14 columns are formed. In the example illustrated, three columns of the 6.5th column, the 0th (7th) column, and the 0.5th column continuous in the circumferential direction are the stretch assisting columns. In the contraction assisting region 24, at least one or preferably a plurality of contraction assisting columns each of which includes a larger number of the hooking portions 22 than the crossing portions 21 in the axial direction (vertical direction) of the stent 2 are disposed. In the example illustrated, six columns of the 1st column to the 6th column are the contraction assisting columns. The 1.5th column, the 2.5th column, the 3.5th column, the 4.5th column, and the 5.5th column formed between the respective contraction assisting columns by the crossing portions 21 are separate stretch assisting columns that are not continuous in the circumferential direction and thus do not constitute the stretch assisting region 23. In this manner, the contraction assisting region 24 including the plurality of contraction assisting columns is formed in a region in the circumferential direction excluding the stretch assisting region 23 including the at least three continuously-disposed stretch assisting columns. In a manner similar to FIG. 4, when the stent 2 is equally divided in the circumferential direction by a plane including the axis of the stent 2 and a first equally-divided region includes the at least three continuously-disposed stretch assisting columns (that is, the stretch assisting region 23), a second equally-divided region (including the contraction assisting region 24 only) includes a majority of the hooking portions 22.

The stretch assisting region 23 and the contraction assisting region 24 extend along the axial direction (vertical direction in FIG. 34) of the stent 2 having a tubular shape. The stretch assisting region 23 including a large number of the crossing portions 21 is easy to stretch and hard to contract along the axial direction of the stent 2 having a tubular shape. The contraction assisting region 24 including a large number of the hooking portions 22 is easy to contract and hard to stretch along the axial direction of the stent 2 having a tubular shape. For this reason, when the stent graft 1 is placed at a dilation target site having a curvature, the contraction assisting region 24 which is easy to contract is disposed on the inner side having a large curvature and the stretch assisting region 23 which is easy to stretch is disposed on the outer side having a small curvature, so that the stent graft 1 can be easily curved.

In the seventh embodiment of FIG. 34, all the hooking portions 22 are provided in the contraction assisting region 24. However, in order to produce a significant difference in stretchability between the stretch assisting region 23 and the contraction assisting region 24, it is enough to provide a majority of the hooking portions 22 in the contraction assisting region 24 (or provide less than half of the hooking portions 22 in the stretch assisting region 23). For example, 70% or more of the hooking portions 22 may be provided in the contraction assisting region 24, or 90% or more of the hooking portions 22 may be provided in the contraction assisting region 24.

As described in the first embodiment, the number of the hooking portions 22 is (X−Z)(Y−1), and the number of the crossing portions 21 is XY+(Y−1)Z. In the seventh embodiment of FIG. 34, X=7, Y=11, and Z=1, and thus the number of the hooking portions 22 is (7 −1)(11 −1)=60, and the number of the crossing portions 21 is 7×11+(11 −1)×1=87. Additionally, in the seventh embodiment described above, the number X of the positions (grid points) in the circumferential direction at which the single wire can form the crossing portion 21 or the hooking portion 22 is an odd number (7), and the one stretch assisting region 23 and the one contraction assisting region 24 are disposed along the circumferential direction.

The present disclosure has been described above based on the embodiments. It should be understood by those skilled in the art that the embodiments are examples, that various modifications are possible in the combination of components and processing operations of the embodiments, and that such modifications are also within the scope of the present disclosure.

### REFERENCE SIGNS LIST

1 Stent graft
2 Stent
3 Graft
21 Intersecting portion
22 Interlocking portion
23 Stretch assisting region
24 Contraction assisting region

1. A stent formed by weaving a wire into a tubular shape, the stent comprising:
intersecting portions at which the wire intersects; and
interlocking portions at which the wire interlocks, wherein
at least two stretch assisting columns are continuously disposed in a circumferential direction of the stent, each of the at least two stretch assisting columns including a larger number of the intersecting portions than the interlocking portions in an axial direction of the stent, and
at least one contraction assisting column including a larger number of the interlocking portions than the intersecting portions in the axial direction is disposed.

2. The stent according to claim 1, wherein when the stent is equally divided in the circumferential direction by a plane including an axis of the stent and a first equally-divided region includes the at least two stretch assisting columns continuously disposed, a second equally-divided region includes a majority of the interlocking portions.

3. The stent according to claim 1, wherein at least three of the stretch assisting columns are continuously disposed in the circumferential direction.

4. The stent according to claim 1, wherein
the number of positions in the circumferential direction at which the wire is capable of forming the intersecting portions or the interlocking portions is 6 or greater, and
a plurality of stretch assisting regions are disposed separately from each other along the circumferential direction, each of the plurality of stretch assisting regions including the at least two stretch assisting columns continuously disposed.

5. The stent according to claim 1, wherein when X is the number of positions in the circumferential direction at which the wire is capable of forming the intersecting portions or the interlocking portions, Y is the number of positions in the axial direction at which the wire is capable of forming the intersecting portions or the interlocking portions, and Z is the number of times the wire is woven back and forth in the axial direction, the number of the interlocking portions is (X−Z)(Y−1), and the number of the intersecting portions is XY+(Y−1)Z.

6. The stent according to claim 1, wherein
a contraction assisting region including a plurality of the contraction assisting columns is formed in a region in the circumferential direction excluding a stretch assisting region including the at least two stretch assisting columns continuously disposed, and
70% or more of the interlocking portions are provided in the contraction assisting region.

7. The stent according to claim 6, wherein 90% or more of the interlocking portions are provided in the contraction assisting region.

8. The stent according to claim 7, wherein all the interlocking portions are provided in the contraction assisting region.

9. The stent according to claim 1, wherein
the wire includes a first wire and a second wire, and
a first stretch assisting region and a second stretch assisting region overlap with each other, the first stretch assisting region including the at least two stretch assisting columns continuously disposed in the first wire, the second stretch assisting region including the at least two stretch assisting columns continuously disposed in the second wire.

10. A stent graft comprising:
a stent formed by weaving a wire into a tubular shape; and
a graft covering the stent, wherein
the stent includes
intersecting portions at which the wire intersects, and
interlocking portions at which the wire interlocks,
at least two stretch assisting columns are continuously disposed in a circumferential direction of the stent, each of the at least two stretch assisting columns including a larger number of the intersecting portions than the interlocking portions in an axial direction of the stent, and
at least one contraction assisting column including a larger number of the interlocking portions than the intersecting portions in the axial direction is disposed.
